(19) United States
(12) Patent Application Publication
(10) Pub. No.: US 2014/0227250 A1
(43) Pub. Date: Aug. 14, 2014

(54) STABLE FORMULATIONS OF ANTIBODIES TO TSLP

(71) Applicant: MERCK SHARP & DOHME CORP., Rahway, NJ (US)

(72) Inventors: Yunsong Li, North Brunswick, NJ (US); Valentyn Antochshuk, Cranford, NJ (US); Anita Dabbara, Princeton Junction, NJ (US); Angela Mohs, East Brunswick, NJ (US); Alexandre Ambrogelly, Westfield, NJ (US)

(73) Assignee: MERCK SHARP & DOHME CORP., Rahway, NJ (US)

(21) Appl. No.: 13/972,157

(22) Filed: Aug. 21, 2013

Related U.S. Application Data
(60) Provisional application No. 61/692,257, filed on Aug. 23, 2012.

(51) Int. Cl. A61K 39/395; C07K 16/24

(52) U.S. Cl. CPC A61K 39/39591; C07K 16/24
USPC 424/133.1

(57) ABSTRACT

The present invention relates to stable formulations of antibodies against human TSLP, or antigen binding fragments thereof.

### FIELD OF THE INVENTION

The present invention relates to stable formulations of antibodies against TSLP, or antigen binding fragments thereof.

### BACKGROUND OF THE INVENTION

TSLP is a cytokine that plays a key role in the initiation, propagation and maintenance of allergic inflammatory responses associated with atopic dermatitis, asthma and food allergies. Genetic evidence demonstrates a strong association of DNA variants and single nucleotide polymorphisms (SNPs) within or near the TSLP gene with atopic dermatitis, asthma and other allergic inflammatory traits. Overall, TSLP is a well-validated target for the development of antibody therapeutics for allergic diseases. TSLP blocking antibodies have been discovered and developed for use to treat allergic diseases such as asthma and atopic dermatitis.

Antibodies for use in human subjects must be stored prior to use, and transported to the point of administration. Reproducibly attaining a desired level of antibody drug in a subject requires that the drug be stored in a formulation that maintains colloidal, biophysical and biochemical stability of the drug, as well as bioactivity. The need exists for stable formulations of anti-TSLP antibodies for pharmaceutical use. Preferably, such formulations will exhibit a long half-life, be stable when stored and transported, and will be amenable to administration at high concentrations, e.g. for use in subcutaneous administration, as well as low concentrations, e.g. for intravenous administration.

### SUMMARY OF THE INVENTION

The present invention relates to stable pharmaceutical formulations of antibodies against TSLP, or antigen binding fragments thereof. The present invention further provides methods for treating allergic diseases, such as asthma and atopic dermatitis, with stable formulations of antibodies against TSLP, or antigen binding fragments thereof.

In certain embodiments, the invention relates to a pharmaceutical formulation of an anti-TSLP antibody, or antigen binding fragment thereof, comprising: a) said anti-TSLP antibody or antigen binding fragment thereof; b) a histidine buffer at a pH range of about pH 5.0-6.0; and (c) a surfactant. Examples of surfactants include polysorbate 80, polysorbate 20 and pluronic F68. In one embodiment, the surfactant is polysorbate 80 at a concentration of at least 0.01% compared to the antibody or antigen binding fragment thereof. In one embodiment, the surfactant is polysorbate 80 at a concentration of 0.01% to 0.10% compared to the antibody or antigen binding fragment thereof. In another embodiment, the surfactant is polysorbate 20 at a concentration of at least 0.01% compared to the antibody or antigen binding fragment thereof. In one embodiment, the surfactant is polysorbate 20 at a concentration of 0.01% to 0.10% compared to the antibody or antigen binding fragment thereof. In another embodiment, the surfactant is pluronic F68 at a concentration of at least 0.10% compared to the antibody or antigen binding fragment thereof. In one embodiment, the surfactant is pluronic F68 at a concentration of 0.10% to 1.0% compared to the antibody or antigen binding fragment thereof. In some embodiments, the formulation further comprises an ionic or a non-ionic stabilizer (which may act as a tonicyfing agent). Examples of non-ionic stabilizers include sucrose, sorbitol, mannitol and trehalose. In one embodiment, the stabilizer is sucrose at a concentration of about 7% compared to the antibody or antigen binding fragment thereof. In another embodiment, the stabilizer is sorbitol at a concentration of about 5% compared to the antibody or antigen binding fragment thereof. In certain embodiments, the formulation has a pH between 5.0 and 6.0 when reconstituted. In one embodiment, the formulation has a pH of about 5.5 when reconstituted.

In certain embodiments, the invention relates to a pharmaceutical formulation of an anti-TSLP antibody, or antigen binding fragment thereof, comprising: a) said anti-TSLP antibody, or antigen binding fragment thereof; b) histidine buffer; c) a surfactant; and d) sucrose. In one embodiment, said formulation is a lyophilized formulation that is intended for reconstitution with sterile water for injection. Examples of surfactants include polysorbate 80, polysorbate 20 and pluronic F68. In one embodiment, the surfactant is polysorbate 80 at a concentration of at least 0.01% compared to the antibody or antigen binding fragment thereof. In another embodiment, the surfactant is polysorbate 20 at a concentration of at least 0.01% compared to the antibody or antigen binding fragment thereof. In another embodiment, the surfactant is pluronic F68 at a concentration of at least 0.10% compared to the antibody or antigen binding fragment thereof.

In certain embodiments, the invention relates to a pharmaceutical formulation of an anti-TSLP antibody, or antigen binding fragment thereof, comprising: a) said anti-TSLP antibody, or antigen binding fragment thereof; b) histidine buffer; c) polysorbate 80; and d) sucrose. In one embodiment, said formulation is a lyophilized formulation that is intended for reconstitution with sterile water for injection.

In certain embodiments, the formulation has a pH between 5.0 and 6.0 when reconstituted. In one embodiment, the formulation has a pH of about 5.5 when reconstituted.

In certain embodiments, the lyophilized formulation enables reconstitution of the antibody, or antigen binding fragment thereof, at a concentration of between about 25 mg/mL and 100 mg/mL. In one embodiment, the lyophilized formulation is for reconstitution of the antibody at 40 mg/mL. In another embodiment, the lyophilized formulation is for reconstitution of the antibody at 100 mg/mL.

In certain embodiments, polysorbate 80 is present at a weight ratio of more than 0.01% compared to the antibody or antigen binding fragment thereof. In one embodiment, polysorbate 80 is present at approximately 0.02% compared to the antibody or antigen binding fragment thereof.

In certain embodiments, sucrose is present at a weight ratio of approximately 7% (7.25%) compared to the antibody, or antigen binding fragment thereof.

In yet additional embodiments, the invention relates to a lyophilized pharmaceutical formulation of an anti-TSLP antibody, or antigen binding fragment thereof, made by lyophilizing an aqueous solution comprising: a) 40 mg/mL anti-antibody, or antigen binding fragment thereof; b) about 70 mg/mL sucrose; c) about 0.2 mg/mL polysorbate 80; and d) about 10 mM histidine buffer at pH 5.0-6.0. In one embodiment, the formulation is for reconstitution at a concentration of 40 mg/mL for intravenous administration.

In other embodiments, the invention relates to a lyophilized pharmaceutical formulation of an anti-TSLP antibody, or antigen binding fragment thereof, made by lyophilizing an aqueous solution comprising: a) 40 mg/mL anti-antibody, or antigen binding fragment thereof; b) about 28-29 mg/mL sucrose; c) about 0.08 mg/mL polysorbate 80; and d) about 4 mM histidine buffer at pH 5.0-6.0. In one embodiment, the formulation is for reconstitution at a concentration of 100 mg/mL for subcutaneous administration.

In yet additional embodiments, the invention relates to a lyophilized pharmaceutical formulation of an anti-TSLP antibody, or antigen binding fragment thereof, that when reconstituted comprises: a) 40-100 mg/mL anti-antibody, or antigen binding fragment thereof; b) about 7% sucrose; c) about 0.02% polysorbate 80; and d) about 10 mM histidine buffer at pH 5.5. In one embodiment, the reconstituted formulation comprises 40 mg/mL and the formulation is for intravenous administration. In one embodiment, the reconstituted formulation comprises 100 mg/mL and the formulation is for subcutaneous administration. In one embodiment, wherein the formulation comprises 100 mg/mL, the formulation comprises a viscosity of less than 4 cP.

In one embodiment, the formulation is stable at a temperature of about ° 5 C. to about 25° C. for at least 12 months, 18 months, 24 months or 36 months. In one embodiment, the formulation is stable at a temperature of about ° 25 C. for at least 24 months. In another embodiment, the formulation is stable at a temperature of about ° 25 C. for at least 36 months. In one embodiment, the formulation is stable following at least 10 cycles of freezing and thawing. In one embodiment, the formulation comprises ≧98.5% monomer of anti-TSLP when stored for at least 12 months at a temperature of about ° 5 C. to about 25° C. In one embodiment, the formulation comprises ≧98.5% monomer of anti-TSLP when stored for at least 12 months at a temperature of about ° 25° C. In one embodiment, the formulation comprises ≧95.0% monomer of anti-TSLP when stored for at least 36 months at a temperature of about ° 5 C. to about 25° C. In one embodiment, the formulation comprises ≧95.0% monomer of anti-TSLP when stored for at least 36 months at a temperature of about 25° C. In one embodiment, the % monomer of anti-TSLP is measured by SEC-HPLC. In one embodiment, the formulation has a viscosity of less than 4 cP at 20° C. when the antibody is present at a concentration of 100 mg/mL (as measured by a MiniVis II Viscometer (Grabner Instruments).

In yet additional embodiments, the invention relates to a method of treating an allergic disease in a mammalian subject in need thereof comprising: administering an effective amount of any of the formulations described herein.

In any of the above embodiments, the anti-TSLP antibody can comprise a light chain comprising the CDR sequences of SEQ ID NO:3, SEQ ID NO:4, SEQ ID NO:5, and a heavy chain comprising the CDR sequences of SEQ ID NO:6, SEQ ID NO:7 and SEQ ID NO:8. In one embodiment, the formulation comprises the light chain of SEQ ID NO:1 and the heavy chain of SEQ ID NO:2.

### BRIEF DESCRIPTION OF THE DRAWINGS

FIG. 1. Analysis of various buffer conditions after 8 weeks (IEX HPLC) and 13 weeks (SEC-HPLC and RP-HPLC) at 40° C.

FIG. 2. Comparison of aTSLP stability time courses in citrate pH 5.0, acetate pH 5.0 and 10 mM histidine pH 5.5 (Buffers B4, B5 and B6) up to 8 weeks (IEX-HPLC) and up to 13 weeks (SEC-HPLC and RP-HPLC) at 40° C.

FIG. 3. Analysis of various buffer conditions after 13 weeks (IEX-HPLC, SEC-HPLC and RP-HPLC) at 25° C.

FIG. 4. Comparison of aTSLP stability time courses in citrate pH 5.0, acetate pH 5.0 and 10 mM histidine pH 5.5 (Buffers B4, B5 and B6) after 13 weeks (IEX-HPLC, SEC-HPLC and RP-HPLC) at 25° C.

FIG. 5. Analysis of various buffer conditions after 13 weeks (IEX-HPLC, SEC-HPLC and RP-HPLC) at 4° C.

FIG. 6. Comparison of stability data at 40° C. for histidine buffer at pH 5.5 and pH 6.0 after 8 weeks (IEX-HPLC) and after 13 weeks (SEC-HPLC and RP-HPLC). Red and blue traces correspond to buffer at pH 5.5 and pH 6.0, respectively.

FIG. 7. Comparison of stability data at 25° C. for histidine buffer at pH 5.5 and pH 6.0 after 13 weeks. Red and blue traces correspond to buffer at pH 5.5 and pH 6.0, respectively.

FIG. 8. Turbidity upon shaking of anti-TSLP. (Note: A320 nm (C1 stressed) was not measured. Shown data is based on observation that the solution appeared to be as turbid as C0 stressed.)

FIG. 9. HP-SEC % monomer stability plot for anti-TSLP Powder for Injection, batch NB-liyun-0321710-0010 at various stability conditions. Solid lines fitted to the data are linear trends up to 12 months. The acceptance criteria for Monomer % is ≧95.0% (dashed line).

FIG. 10. HP-IEX % main species stability plot for anti-TSLP Powder for Injection, batch NB-liyun-0321710-0010 at various stability conditions. Solid lines fitted to the data are linear trends up to 12 months.

FIG. 11. HP-IEX % acidic species plot for anti-TSLP Powder for Injection, batch NB-liyun-0321710-0010 at various stability conditions. Solid lines fitted to the data are linear trends up to 12 months.

FIG. 12. HP-IEX % basic species plot for anti-TSLP Powder for Injection, batch NB-liyun-0321710-0010 at various stability conditions. Solid lines fitted to the data are linear trends up to 12 months.

FIG. 13. HP-SEC % monomer stability plot for anti-TSLP Powder for Injection, batch 89782-101 at various stability conditions. Solid lines fitted to the data are linear trends up to 12 months. The acceptance criteria for Monomer % is ≧95.0% (dashed line).

FIG. 14. HP-IEX % main species stability plot for anti-TSLP Powder for Injection, batch 89782-101 at various stability conditions. Solid lines fitted to the data are linear trends up to 12 months.

FIG. 15. HP-IEX % acidic species plot for anti-TSLP Powder for Injection, batch 89782-101 at various stability conditions. Solid lines fitted to the data are linear trends up to 12 months.

FIG. 16. HP-IEX % basic species plot for anti-TSLP Powder for Injection, batch 89782-101 at various stability conditions. Solid lines fitted to the data are linear trends up to 12 months.

### DETAILED DESCRIPTION

The present invention provides formulations of anti-TSLP antibodies and uses thereof for treating allergic diseases, such as asthma and atopic dermatitis.

In accordance with the present invention there may be employed conventional molecular biology, microbiology, protein expression and purification, antibody, and recombinant DNA techniques within the skill of the art. Such techniques are explained fully in the literature. See, e.g., Sambrook et al. (2001) Molecular Cloning: A Laboratory Manual. 3^{rd} ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N J; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.; Nucleic Acid Hybridization, Hames & Higgins eds. (1985); Transcription And Translation, Hames & Higgins, eds. (1984); Animal Cell Culture Freshney, ed. (1986); Immobilized Cells And Enzymes, IRL Press (1986); Perbal, A Practical Guide To Molecular Cloning (1984); and Harlow and Lane. Antibodies: A Laboratory Manual (Cold Spring Harbor Laboratory Press: 1988).

### I. DEFINITIONS

As used herein, the term “antibody” refers to any form of antibody that exhibits the desired biological activity. Thus, it is used in the broadest sense and specifically covers monoclonal antibodies (including full length monoclonal antibodies), polyclonal antibodies, multispecific antibodies (e.g., bispecific antibodies), chimeric antibodies, humanized antibodies, fully human antibodies, etc. so long as they exhibit the desired biological activity.

As used herein, the terms “TSLP binding fragment,” “antigen binding fragment thereof,” “binding fragment thereof” or “fragment thereof” encompass a fragment or a derivative of an antibody that still substantially retains its biological activity of binding to antigen (human TSLP) and inhibiting its activity (e.g., blocking the binding of TSLP to TSLPR). Therefore, the term “antibody fragment” or TSLP binding fragment refers to a portion of a full length antibody, generally the antigen binding or variable region thereof. Examples of antibody fragments include Fab, Fab′, F(ab′)₂, and Fv fragments; diabodies; linear antibodies; single-chain antibody molecules, e.g., sc-Fv; and multispecific antibodies formed from antibody fragments. Typically, a binding fragment or derivative retains at least 10% of its TSLP inhibitory activity. Preferably, a binding fragment or derivative retains at least 25%, 50%, 60%, 70%, 80%, 90%, 95%, 99% or 100% (or more) of its TSLP inhibitory activity, although any binding fragment with sufficient affinity to exert the desired biological effect will be useful. It is also intended that a TSLP binding fragment can include variants having conservative amino acid substitutions that do not substantially alter its biologic activity.

The phrase “consists essentially of,” or variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a binding compound that consists essentially of a recited amino acid sequence may also include one or more amino acids, including substitutions of one or more amino acid residues, that do not materially affect the properties of the binding compound.

### Pharmaceutical Composition Definitions

The term “bulking agents” comprise agents that provide the structure of the freeze-dried product. Common examples used for bulking agents include manitol, glycine, lactose and sucrose. In addition to providing a pharmaceutically elegant cake, bulking agents may also impart useful qualities in regard to modifying the collapse temperature, providing freeze-thaw protection, and enhancing the protein stability over long-term storage. These agents can also serve as tonicity modifiers.

The term “buffer” encompasses those agents which maintain the solution pH in an acceptable range prior to lyophilization and may include succinate (sodium or potassium), histidine, phosphate (sodium or potassium), Tris(tris (hydroxymethyl)aminomethane), diethanolamine, citrate (sodium) and the like. The buffer of this invention has a pH in the range from about 5.0 to about 6.0; and preferably has a pH of about 5.5.

The term “cryoprotectants” generally includes agents which provide stability to the protein against freezing-induced stresses, presumably by being preferentially excluded from the protein surface. They may also offer protection during primary and secondary drying, and long-term product storage. Examples are polymers such as dextran and polyethylene glycol; sugars such as sucrose, glucose, trehalose, and lactose; surfactants such as polysorbates; and amino acids such as glycine, arginine, and serine.

The terms “lyophilization,” “lyophilized,” and “freeze-dried” refer to a process by which the material to be dried is first frozen and then the ice or frozen solvent is removed by sublimation in a vacuum environment. An excipient may be included in pre-lyophilized formulations to enhance stability of the lyophilized product upon storage.

The term “lyoprotectant” includes agents that provide stability to the protein during the drying or ‘dehydration’ process (primary and secondary drying cycles), presumably by providing an amorphous glassy matrix and by binding with the protein through hydrogen bonding, replacing the water molecules that are removed during the drying process. This helps to maintain the protein conformation, minimize protein degradation during the lyophilization cycle and improve the long-term product stability. Examples include polyols or sugars such as sucrose and trehalose.

The term “pharmaceutical formulation” refers to preparations which are in such form as to permit the active ingredients to be effective, and which contains no additional components which are toxic to the subjects to which the formulation would be administered.

“Pharmaceutically acceptable” excipients (vehicles, additives) are those which can reasonably be administered to a subject mammal to provide an effective dose of the active ingredient employed.

“Reconstitution time” is the time that is required to rehydrate a lyophilized formulation with a solution to a particle-free clarified solution.

A “stable” formulation is one in which the protein therein essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Stability can be measured at a selected temperature for a selected time period. For example, in one embodiment, a “stable” lyophilized antibody formulation is a lyophilized antibody formulation with no significant changes observed at a refrigerated temperature (2-8° C.) for at least 12 months, preferably 2 years, and more preferably 3 years. In another embodiment, “stable” lyophilized antibody formulation is a lyophilized antibody formulation with no significant changes observed at or at room temperature (23-27° C.) for at least 3 months, 6 months, 1 year, 2 years or 3 years. The criteria for stability are as follows. No more than 10%, preferably 5%, of antibody monomer is degraded as measured by SEC-HPLC. The rehydrated solution is colorless, or clear to slightly opalescent by visual analysis. The concentration, pH and osmolality of the formulation have no more than ^{+/-}10% change. Potency is within 70-130, preferably 80-120% of the control. No more than 10%, preferably 5% of clipping is observed. No more than 10%, preferably 5% of aggregation is formed.

An antibody “retains its physical stability” in a pharmaceutical formulation if it shows no significant increase of aggregation, precipitation and/or denaturation upon visual examination of color and/or clarity, or as measured by UV light scattering, size exclusion chromatography (SEC) and dynamic light scattering. The changes of protein conformation can be evaluated by fluorescence spectroscopy, which determines the protein tertiary structure, and by FTIR spectroscopy, which determines the protein secondary structure.

An antibody “retains its chemical stability” in a pharmaceutical formulation, if it shows no significant chemical alteration. Chemical stability can be assessed by detecting and quantifying chemically altered forms of the protein. Degradation processes that often alter the protein chemical structure include hydrolysis or clipping (evaluated by methods such as size exclusion chromatography and SDS-PAGE), oxidation (evaluated by methods such as by peptide mapping in conjunction with mass spectroscopy or MALDI/TOF/MS), deamidation (evaluated by methods such as ion-exchange chromatography, capillary isoelectric focusing, peptide mapping, isoaspartic acid measurement), and isomerization (evaluated by measuring the isoaspartic acid content, peptide mapping, etc.).

An antibody “retains its biological activity” in a pharmaceutical formulation, if the biological activity of the antibody at a given time is within a predetermined range of the biological activity exhibited at the time the pharmaceutical formulation was prepared. The biological activity of an antibody can be determined, for example, by an antigen binding assay.

The term “isotonic” means that the formulation of interest has essentially the same osmotic pressure as human blood. Isotonic formulations will generally have an osmotic pressure from about 270-328 mOsm. Slightly hypotonic pressure is 250-269 and slightly hypertonic pressure is 328-350 mOsm. Osmotic pressure can be measured, for example, using a vapor pressure or ice-freezing type osmometer.

Tonicity Modifiers: Salts (NaCl, KCl, MgCl₂, CaCl₂, etc) are used as tonicity modifiers to control osmotic pressure. In addition, cryprotecants/lyoprotectants and/or bulking agents such as sucrose, mannitol, glycine etc. can serve as tonicity modifiers.

### Analytical Methods

Analytical methods suitable for evaluating the product stability include size exclusion chromatography (SEC), dynamic light scattering test (DLS), differential scanning calorimetery (DSC), iso-asp quantification, potency, UV at 340 nm, UV spectroscopy, and FTIR. SEC (J. Pharm. Scien., 83:1645-1650, (1994); Pharm. Res., 11:485 (1994); J. Pharm. Bio. Anal., 15:1928 (1997); J. Pharm. Bio. Anal., 14:1133-1140 (1986)) measures percent monomer in the product and gives information of the amount of soluble aggregates. DSC (Pharm. Res., 15:200 (1998); Pharm. Res., 9:109 (1982)) gives information of protein denaturation temperature and glass transition temperature. DLS (American Lab., November (1991)) measures mean diffusion coefficient, and gives information of the amount of soluble and insoluble aggregates. UV at 340 nm measures scattered light intensity at 340 nm and gives information about the amounts of soluble and insoluble aggregates. UV spectroscopy measures absorbance at 278 nm and gives information of protein concentration. FTIR (Eur. J. Pharm. Biopharm., 45:231 (1998); Pharm. Res., 12:1250 (1995); J. Pharm. Scien., 85:1290 (1996); J. Pharm. Scien., 87:1069 (1998)) measures IR spectrum in the amide one region, and gives information of protein secondary structure.

The iso-asp content in the samples is measured using the Isoquant Isoaspartate Detection System (Promega). The kit uses the enzyme Protein Isoaspartyl Methyltransferase (PIMT) to specifically detect the presence of isoaspartic acid residues in a target protein. PIMT catalyzes the transfer of a methyl group from S-adenosyl-L-methionine to isoaspartic acid at the .alpha.-carboxyl position, generating S-adenosyl-L-homocysteine (SAH) in the process. This is a relatively small molecule, and can usually be isolated and quantitated by reverse phase HPLC using the SAH HPLC standards provided in the kit.

The potency or bioidentity of an antibody can be measured by its ability to bind to its antigen. The specific binding of an antibody to its antigen can be quantitated by any method known to those skilled in the art, for example, an immunoassay, such as ELISA (enzyme-linked immunosorbant assay).

A “reconstituted” formulation is one that has been prepared by dissolving a lyophilized protein formulation in a diluent such that the protein is dispersed in the reconstituted formulation. The reconstituted formulation is suitable for administration, e.g. parenteral administration), and may optionally be suitable for subcutaneous administration.

### Anti-TSLP Antibodies

Any anti-TSLP antibody could be used in the formulations on the invention. In preferred embodiments, the anti-TSLP antibodies to be used with the claimed formulations are the ones described in WO2008/076321 and WO2011/056772.

### Biological Activity of Humanized Anti-TSLP

Formulations of the present invention include anti-TSLP antibodies and fragments thereof that are biologically active when reconstituted. As used herein, the term “biologically active” refers to an antibody or antibody fragment that is capable of binding the desired the antigenic epitope and directly or indirectly exerting a biologic effect. Typically, these effects result from the failure of TSLP to bind its receptor. As used herein, the term “specific” refers to the selective binding of the antibody to the target antigen epitope. Antibodies can be tested for specificity of binding by comparing binding to TSLP to binding to irrelevant antigen or antigen mixture under a given set of conditions.

### Lyophilized Pharmaceutical Compositions

Lyophilized formulations of therapeutic proteins provide several advantages. Lyophilized formulations in general offer better chemical stability than solution formulations, and thus increased half-life. A lyophilized formulation may also be reconstituted at different concentrations depending on clinical factors, such as route of administration or dosing. For example, a lyophilized formulation may be reconstituted at a high concentration (i.e. in a small volume) if necessary for subcutaneous administration, or at a lower concentration if administered intravenously. High concentrations may also be necessary if high dosing is required for a particular subject, particularly if administered subcutaneously where injection volume must be minimized. One such lyophilized antibody formulation is disclosed at U.S. Pat. No. 6,267,958, which is hereby incorporated by reference in its entirety. Lyophilized formulations of another therapeutic protein are disclosed at U.S. Pat. No. 7,247,707, which is hereby incorporated by reference in its entirety.

Typically the lyophilized formulation is prepared in anticipation of reconstitution at high concentration of drug product (DP, in an exemplary embodiment humanized anti-TSLP antibody, or antigen binding fragment thereof), i.e. in anticipation of reconstitution in a low volume of water. Subsequent dilution with water or isotonic buffer can then readily be used to dilute the DP to a lower concentration. Typically, excipients are included in a lyophilized formulation of the present invention at levels that will result in a roughly isotonic formulation when reconstituted at high DP concentration, e.g. for subcutaneous administration. Reconstitution in a larger volume of water to give a lower DP concentration will necessarily reduce the tonicity of the reconstituted solution, but such reduction may be of little significance in non-subcutaneous, e.g. intravenous, administration. If isotonicity is desired at lower DP concentration, the lyophilized powder may be reconstituted in the standard low volume of water and then further diluted with isotonic diluent, such as 0.9% sodium chloride.

In one embodiment of the present invention, anti-TSLP antibody (or antigen binding fragment thereof) is formulated as a lyophilized powder for intravenous administration. In another embodiment of the present invention, anti-TSLP antibody (or antigen binding fragment thereof) is formulated as a lyophilized powder for subcutaneous administration. In certain embodiments, the antibody (or antigen binding fragment thereof) is provided at about 40-300 mg/vial, and is reconstituted with sterile water for injection prior to use. In other embodiments, the antibody (or antigen binding fragment thereof) is provided at about 40-100 mg/vial, and is reconstituted with sterile water for injection prior to use. The target pH of the reconstituted formulation is 5.5. In various embodiments, the lyophilized formulation of the present invention enables reconstitution of the anti-TSLP antibody to high concentrations, such as about 20, 25, 30, 40, 50, 60, 75, 100, 150, 200, 250 or more mg/mL.

The present invention provides in certain embodiments, a lyophilized formulation comprising humanized anti-TSLP antibody, a histidine buffer at about pH 5.5, or at about pH 5.0, for example at about 5.1, 5.2, 5.3, 5.4, 5.6, 5.7, 5.8, 5.9, or 6.0.

When a range of pH values is recited, such as “a pH between pH 5.5 and 6.0,” the range is intended to be inclusive of the recited values. Unless otherwise indicated, the pH refers to the pH after reconstitution of the lyophilized formulations of the present invention. The pH is typically measured at 25° C. using standard glass bulb pH meter. As used herein, a solution comprising “histidine buffer at pH X” refers to a solution at pH X and comprising the histidine buffer, i.e. the pH is intended to refer to the pH of the solution.

Lyophilized formulations are by definition essentially dry, and thus the concept of concentration is not useful in describing them. Describing a lyophilized formulation in the terms of the weight of the components in a unit dose vial is more useful, but is problematic because it varies for different doses or vial sizes. In describing the lyophilized formulations of the present invention, it is useful to express the amount of a component as the ratio of the weight of the component compared to the weight of the drug substance (DS) in the same sample (e.g. a vial). This ratio may be expressed as a percentage. Such ratios reflect an intrinsic property of the lyophilized formulations of the present invention, independent of vial size, dosing, and reconstitution protocol.

In other embodiments, the lyophilized formulation of anti-TSLP antibody, or antigen binding fragment, is defined in terms of the pre-lyophilization solution used to make the lyophilized formulation, such as the pre-lyophilization solution. In one embodiment the pre-lyophilization solution comprises antibody, or antigen-binding fragment thereof, at a concentration of about 40 mg/mL. Such pre-lyophilization solutions may be at about pH 5.5, or range from about pH 5.0 to about 6.0.

In yet other embodiments, the lyophilized formulation of anti-TSLP antibody, or antigen binding fragment, is defined in terms of the reconstituted solution generated from the lyophilized formulation. Reconstituted solutions may comprise antibody, or antigen-binding fragment thereof, at concentrations of about 10, 15, 20, 25, 30, 40, 50, 60, 75, 80, 90 or 100 mg/mL or higher concentrations such as 150 mg/mL, 200 mg/mL, 250 mg/mL, or up to about 300 mg/mL. In one embodiment, the reconstituted formulation may comprise 40 mg/mL of the antibody, or antigen-binding fragment thereof. In another embodiment, the reconstituted formulation may comprise 100 mg/mL of the antibody, or antigen-binding fragment thereof. Such reconstituted solutions may be at about pH 5.5, or range from about pH 5.0 to about 6.0.

The lyophilized formulations of the present invention are formed by lyophilization (freeze-drying) of a pre-lyophilization solution. Freeze-drying is accomplished by freezing the formulation and subsequently subliming water at a temperature suitable for primary drying. Under this condition, the product temperature is below the eutectic point or the collapse temperature of the formulation. Typically, the shelf temperature for the primary drying will range from about −30 to 25° C. (provided the product remains frozen during primary drying) at a suitable pressure, ranging typically from about 50 to 250 mTorr. The formulation, size and type of the container holding the sample (e.g., glass vial) and the volume of liquid will dictate the time required for drying, which can range from a few hours to several days (e.g. 40-60 hrs). A secondary drying stage may be carried out at about 0-40° C., depending primarily on the type and size of container and the type of protein employed. The secondary drying time is dictated by the desired residual moisture level in the product and typically takes at least about 5 hours. Typically, the moisture content of a lyophilized formulation is less than about 5%, and preferably less than about 3%. The pressure may be the same as that employed during the primary drying step. Freeze-drying conditions can be varied depending on the formulation and vial size.

In some instances, it may be desirable to lyophilize the protein formulation in the container in which reconstitution of the protein is to be carried out in order to avoid a transfer step. The container in this instance may, for example, be a 3, 5, 10, 20, 50 or 100 cc vial.

The lyophilized formulations of the present invention are reconstituted prior to administration. The protein may be reconstituted at a concentration of about 10, 15, 20, 25, 30, 40, 50, 60, 75, 80, 90 or 100 mg/mL or higher concentrations such as 150 mg/mL, 200 mg/mL, 250 mg/mL, or 300 mg/mL up to about 500 mg/mL. High protein concentrations are particularly useful where subcutaneous delivery of the reconstituted formulation is intended. However, for other routes of administration, such as intravenous administration, lower concentrations of the protein may be desired (e.g. from about 5-50 mg/mL).

Reconstitution generally takes place at a temperature of about 25° C. to ensure complete hydration, although other temperatures may be employed as desired. The time required for reconstitution will depend, e.g., on the type of diluent, amount of excipient(s) and protein. Exemplary diluents include sterile water, bacteriostatic water for injection (BWFI), a pH buffered solution (e.g. phosphate-buffered saline), sterile saline solution, Ringer's solution or dextrose solution.

Various literature references are available to facilitate selection of pharmaceutically acceptable carriers or excipients. See, e.g., Remington's Pharmaceutical Sciences and U.S. Pharmacopeia: National Formulary, Mack Publishing Company, Easton, Pa. (1984); Hardman et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.

Toxicity is a consideration in selecting the proper dosing of a therapeutic agent, such as a humanized anti-TSLP antibody (or antigen binding fragment thereof). Toxicity and therapeutic efficacy of the antibody compositions, administered alone or in combination with an immunosuppressive agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio of LD₅₀ to ED₅₀. Antibodies exhibiting high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized.

Suitable routes of administration may, for example, include parenteral delivery, including intramuscular, intradermal, subcutaneous, intramedullary injections, as well as intrathecal, direct intraventricular, intravenous, intraperitoneal. Drugs can be administered in a variety of conventional ways, such as intraperitoneal, parenteral, intraarterial or intravenous injection. Modes of administration in which the volume of solution must be limited (e.g. subcutaneous administration) require that a lyophilized formulation to enable reconstitution at high concentration.

Alternately, one may administer the antibody in a local rather than systemic manner, for example, via injection of the antibody directly into a pathogen-induced lesion characterized by immunopathology, often in a depot or sustained release formulation. Furthermore, one may administer the antibody in a targeted drug delivery system, for example, in a liposome coated with a tissue-specific antibody, targeting, for example, pathogen-induced lesion characterized by immunopathology. The liposomes will be targeted to and taken up selectively by the afflicted tissue.

Selecting an administration regimen for a therapeutic depends on several factors, including the serum or tissue turnover rate of the entity, the level of symptoms, the immunogenicity of the entity, and the accessibility of the target cells in the biological matrix. Preferably, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002).

Determination of the appropriate dose is made by the clinician, e.g., using parameters or factors known or suspected in the art to affect treatment or predicted to affect treatment. The appropriate dosage (“therapeutically effective amount”) of the protein will depend, for example, on the condition to be treated, the severity and course of the condition, whether the protein is administered for preventive or therapeutic purposes, previous therapy, the patient's clinical history and response to the protein, the type of protein used, and the discretion of the attending physician. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. The protein is suitably administered to the patient at one time or repeatedly. The protein may be administered alone or in conjunction with other drugs or therapies.

Antibodies, or antibody fragments can be provided by continuous infusion, or by doses at intervals of, e.g., one day, 1-7 times per week, one week, two weeks, three weeks, monthly, bimonthly, etc. A preferred dose protocol is one involving the maximal dose or dose frequency that avoids significant undesirable side effects.

In certain embodiments, dosing will comprise administering to a subject escalating doses of 1.0, 3.0, and 10 mg/kg of the pharmaceutical formulation over the course of treatment. Time courses can vary, and can continue as long as desired effects are obtained.

In certain embodiments, the pharmaceutical formulations of the invention will be administered by intravenous (IV) infusion.

In other embodiments, the pharmaceutical formulations of the invention will be administered by subcutaneous administration. Subcutaneous administration may performed by injected using a syringe, or using other injection devices (e.g. the Injectease® device); injector pens; or needleless devices (e.g. MediJector and BioJector®).

The broad scope of this invention is best understood with reference to the following examples, which are not intended to limit the inventions to the specific embodiments. The specific embodiments described herein are offered by way of example only, and the invention is to be limited by the terms of the appended claims, along with the full scope of equivalents to which such claims are entitled.

### EXAMPLES

### Example 1

### Antibody Production

The anti-TSLP antibody used in the Examples is a humanized monoclonal antibody (mAb) that is described in International Patent Application No. WO2011/056772, which is referred to herein as “aTSLP”. It consists of a human gamma 1 heavy chain and a kappa light chain, and comprises the sequences outlined below.

The light chain comprises the amino acid sequence of SEQ ID NO:1 (EIVLTQSPGT LSLSPGERAT LSCRASQPIS ISVHWYQQKP GQAPRLLIYF ASQSISGIPD RFSGSGSGTD FTLTISRLEP EDFAVYYCQQ TFSLPYTFGQ GTKVEIKRTV AAPSVFIFPP SDEQLKSGTA SVVCLLNNFY PREAKVQWKV DNALQSGNSQ ESVTEQDSKD STYSLSSTLT LSKADYEKHK VYACEVTHQG LSSPVTKSFN RGEC.

The heavy chain comprises the amino acid sequence of SEQ ID NO: 2 (QVQLVQSGAE VKKPGASVKV SCKASGYIFT DYAMHWVRQA PGQGLEWMGT FIPLLDTSDY AQKFQGRVTM TADTSTSTAY MELRSLRSDD TAVYYCARMG VTHSYVMDAW GQGTLVTVSS ASTKGPSVFP LAPSSKSTSG GTAALGCLVK DYFPEPVTVS WNSGALTSGV HTFPAVLQSS GLYSLSSVVT VPSSSLGTQT YICNVNHKPS NTKVDKKVEP KSCDKTHTCP PCPAPELLGG PSVFLFPPKP KDTLMISRTP EVTCVVVDVS HEDPEVKFNW YVDGVEVHNA KTKPREEQYN STYRVVSVLT VLHQDWLNGK EYKCKVSNKA LPAPIEKTIS KAKGQPREPQ VYTLPPSRDE LTKNQVSLTC LVKGFYPSDI AVEWESNGQP ENNYKTTPPV LDSDGSFFLY SKLTVDKSRW QQGNVFSCSV MHEALHNHYT QKSLSLSPGK.

TABLE 1
aTSLP CDR sequences

| | |
|---|---|
| CDR L1 | RASQPISISVH (SEQ ID NO: 3) |
| CDR L2 | FASQSIS (SEQ ID NO: 4) |
| CDR L3 | QQTFSLPYT (SEQ ID NO: 5) |
| CDR H1 | GYIFTDYAMH (SEQ ID NO: 6) |
| CDR H2 | TFIPLLDTSDYAQKFQG (SEQ ID NO: 7) |
| CDR H3 | MGVTHSYVMDA (SEQ ID NO: 8) |

Construction of the Expression Plasmid, pATSLPV1, for Development of the Manufacturing Cell Line

An expression plasmid was constructed for the expression of both the heavy and light antibody chains. The pATSLPV1 expression vector was subsequently used to transfect CHO DXB-11 cells.

The host cell line used for expression of the anti-TSLP antibody, CHO-DXB-11, was obtained from Dr. Larry Chasin at Columbia University.

To establish cell lines producing the anti-TSLP antibody, suspension-adapted CHO-DXB-11 cells were transfected with the plasmid pATSLPV1. The growth properties and antibody production of the candidate clones in suspension culture were monitored. Clone W3-5B was selected as a production candidate, and a Master Seed Bank (MSB) designated as “aTSLP-W3-5B-MSB” was prepared. Cultures established from the MSB have demonstrated stable growth and productivity over an extended culture period of at least 2 months. The MSB was tested for mycoplasma prior to use in the manufacture of drug substance for toxicity studies. The MSB was also used to prepare the Master Cell Bank (MCB).

The Master Cell Bank was prepared under Good Manufacturing Practice (GMP) conditions.

The upstream manufacturing process for producing aTSLP antibody is a suspension cell culture process that uses commercially-available animal component-free medium. Cells from the MCB are propagated and scaled-up to inoculate a 2700-liter bioreactor. Timing of harvest of the production bioreactor is based primarily upon culture viability. At bioreactor harvest, phosphate buffer is added to the bioreactor to maintain pH during cold storage. The contents of the bioreactor are processed by centrifugation, depth filtration to remove intact cells and cell debris and then 0.2 μm filtered into pre-sterilized bags. The clarified broth is stored under refrigerated conditions prior to purification. At the end of fermentation, each batch of unprocessed bulk material is tested for sterility, mycoplasma, and adventitious virus.

The aTSLP antibody is purified using standard procedures. The purified antibody is diafiltered into its formulation buffer through an ultrafiltration/diafiltration step and compounded with excipients into drug substance with the formulations described below. The antibody formulations are then filtered through 0.2 nm filter to control bioburden and subsequently stored at refrigerated conditions (2-8° C.).

### Example 2

### Formulation Studies

### Materials and Methods

The anti-TSLP antibody (“aTSLP”) described in Example 1 was diluted to a final concentration of 1 mg per ml in 12 different formulations which included:
20 mM sodium citrate pH 3.0 (“B1”)
20 mM sodium citrate pH 4.0 (“B2”)
20 mM sodium acetate pH 4.0 (“B3”)
20 mM sodium citrate pH 5.0 (“B4”)
20 mM sodium acetate pH 5.0 (“B5”)
20 mM histidine pH 5.5 (“B6”)
20 mM sodium phosphate pH 6.0 (“B7”)
20 mM histidine pH 6.0 (“B8”)
20 mM sodium phosphate pH 6.5 (“B9”)
20 mM sodium citrate pH 7.0 (“B10”)
20 mM sodium phosphate pH 7.0 (“B11”)
20 mM Tris-HCl pH 8.0 (“B12”)

The anti-TSLP antibody in the various buffer formulations were setup on stability at 2-8° C., 25° C. and 40° C. for up to 3 months. During this time the samples were analyzed at various time points.

### Analytical Procedures

RP-HPLC:

Briefly, samples were run on a Poros R2/10 column (2.1 mm×30.0 mm, cat #1-1112-12) on an Agilent 1200 system. The column was equilibrated with solution A (0.2% TFA in water) and developed with a gradient of solution B (0.2% TFA in 90% acetonitrile), going from 25% B buffer to 60% in 5 minutes at a 2 ml/min flow rate.

SEC-HPLC:

Size exclusion HPLC Chromatography was performed on a GE S200 column (10 mm×300 mm, cat#17-5175-01) on Agilent 1100 systems. Mobile phase for the 5200 column was 1×PBS pH 7.4 (Sigma cat# P-3813). Chromatography was performed at room temperature at a 0.5 ml/min flow rate for 60 min.

IEX-HPLC:

Ion Exchange HPLC was used to evaluate charge heterogeneity of the samples in the course of the study. The samples were run on a Dionex WCX-10 column. The column was equilibrated with 20 mM sodium citrate pH 5.5 and developed with a linear gradient of sodium phosphate at pH 8.0: from 20 to 60.5% B in 15 min. The composition of buffer B was 95 mM NaCl, 20 mM phosphate pH 8.0.

Reducing and Non-Reducing SDS-PAGE:

Reducing and non-reducing SDS-PAGE was used to monitor the presence of small peptides as well as the formation of high molecular weight species in the samples.

Differential Scanning Calorimetry (DSC):

DSC was carried out on VP-DSC microcalorimeter (MicroCal LLC), having a cell volume of 0.142 ml. Protein solutions were heated from 20° C. to 95° C. with a heating rate of 1° C./min. Before starting the temperature scan, the samples were equilibrated at 20° C. for 5 min. One data point was averaged for 16s. All samples were run in duplicate. The analysis of apparent Tₘ was obtained by using the MicroCal Origin 7 software. After subtraction of buffer scans from the protein scans, the scans were normalized for protein concentration. Note that Tₘ has to be considered apparent, since the transitions are not reversible.

Dynamic Light Scattering (DLS):

Dynamic light scattering was carried out on a Malvern Zetasizer Nano ZS, model ZEN3600 (UK). Scattering cells used were from Hellma (Quartz Suprasil, 3 mm light path, fill volume: 45 μL). After passing the cell the intensity of the scattered light was detected at a fixed angle of 90°. An autocorrelator calculates the intensity-intensity autocorrelation function

G(τ)= [symbol] I(t)I(t+τ) [symbol] ,

where τ is the sample time. From the intensity-intensity autocorrelation function the diffusion coefficient is determined via:

G(τ)=A(1+Bexp(−2D,qτ)),

where A is the baseline of the correlation function, B is the intercept of the correlation function and q is the scattering vector: q=(4πn/λ₀)sin(θ/2). n is the refractive index, λ₀ is the wavelength of the laser (633 nm) and θ is the scattering angle(90°). The viscosity and refractice index were assumed to be close to water.

Circular Dichroism Spectroscopy (CD):

CD was performed on a circular dichroism spectropolarimeter (JASCO, Easton, Md.), model JASCO J-810-1505. The ellipticity was monitored with a data pitch of 0.2 nm and a scanning speed of 50 nm/min in the interval of 250 nm-350 nm for both the buffer and the protein solutions. 5 scans were averaged to obtain the final spectrum. Cell cuvettes of 1 mm were used. For data analysis, the buffer scan was substracted from the protein scans. The buffer substracted scans were normalized to molecular rest weight by using the equation:

$${Θ}_{MRW} = \frac{Θ100 M W}{c d {N}_{A}} ,$$

where Θ is the measured ellipticity in degree, c is to protein concentration in mg/ml, d is the path length in cm, MW is the protein molecular weight in Dalton and N_{A} is the number of amino acids. MW and N_{A} used were 148099 Da and 1328, respectively.

### Results

Study Rationale:

Preformulation studies were conducted to determine the optimal buffer conditions to formulate the antibody which is stable over long period of times. For the aTSLP preformulation studies we used five different buffer systems (sodium citrate, sodium acetate, sodium phosphate, histidine and Tris-HCl) with pHs ranging from 3.0 to 8.0. The aTSLP antibody was formulated at 1 mg/ml in the 12 buffers outlined above.

### Biochemical and Biophysical Properties at Initial Time Point

Biochemical and biophysical properties of aTSLP molecule diluted in each of the 12 formulations were assessed at the initial time point.

HPLC:

All samples presented identical IEX-HPLC, SEC-HPLC and RP-HPLC chromatograms at the initial time point.

Near UV CD:

The near UV spectrum of aTSLP is complex. All aTSLP near UV CD spectra overlay for all different buffers. Slight changes are observed within 270 nm-280 nm for aTSLP in citrate buffer at pH 4 compared to other pH buffers.

Dynamic Light Scattering (“DLS”):

Dynamic light scattering has been used to determine the hydrodynamic radius of aTSLP in different formulations. Each sample was rerun 3 times and the experimental error of measurement of one single sample was found to be on the order of ±0.5 nm. The hydrodynamic diameter of aTSLP is similar for most formulations, between 11.5 nm up to 12.8 nm. The hydrodynamic diameter of aTSLP in acetate buffer, pH 4 seems slightly lower (10.2 nm) than in other formulations. There is no indication of aggregate formation in any of the samples.

Differential Scanning Calorimetry:

Differential scanning calorimetry has been determined to evaluate the thermal stability upon heating of aTSLP in different formulations. In general, the unfolding transitions of aTSLP are shifted towards higher temperature with increase in pH, meaning that aTSLP is more stable at higher pH-values. Two well separated unfolding transitions are observed in all formulations, with the exception of citrate pH 3. In citrate buffer at pH 3 aTSLP shows a third, broad unfolding transition. Based on DSC data obtained for other antibodies, the first transition might be unfolding of the CH2-domain of the FC-fragment and the second transition simultaneous unfolding of the FAB-fragment and the CH3-domain of the FC-fragment. A difference in stability of aTSLP is observed between acetate pH 4 and citrate pH 4, which might be attributed to differences in ionic strength. See above for DLS data.

### Stability at 40° C.

The aTSLP monoclonal antibody formulated in the 12 different solutions was analyzed by IEX-HPLC, SEC-HPLC and RP-HPLC. Samples were analyzed at 2, 3, 4, 6 and 8 weeks by IEX-HPLC, SEC-HPLC and RP-HPLC and up to 13 weeks by SEC-HPLC and RP-HPLC.

Comparison of all 12 Formulations Conditions:

An overlay of the chromatographic profiles obtained after up to 13 weeks at 40° C. showed gross differences in IEX-HPLC, SEC-HPLC and RP-HPLC chromatograms.

Samples monitored by IEX-HPLC displayed severe differences in aTSLP surface charge distribution. Expectedly, the most dramatic changes were observed in the most acidic and basic conditions (i.e. citrate pH 3.0 [B1] and Tris-HCl pH 8.0 [B12]). Storage in citrate pH 3.0 [B1] for 8 weeks resulted in a chromatogram presenting severe breakthrough as well as a late eluting peak corresponding to a species with a net increase in positive charges. At the other end of the pH spectrum, storage at 40° C. for 8 weeks in Tris-HCl pH 8.0 [B12] resulted in a decrease in basic variants, dramatic reduction of the main peak and increase in acidic variants.

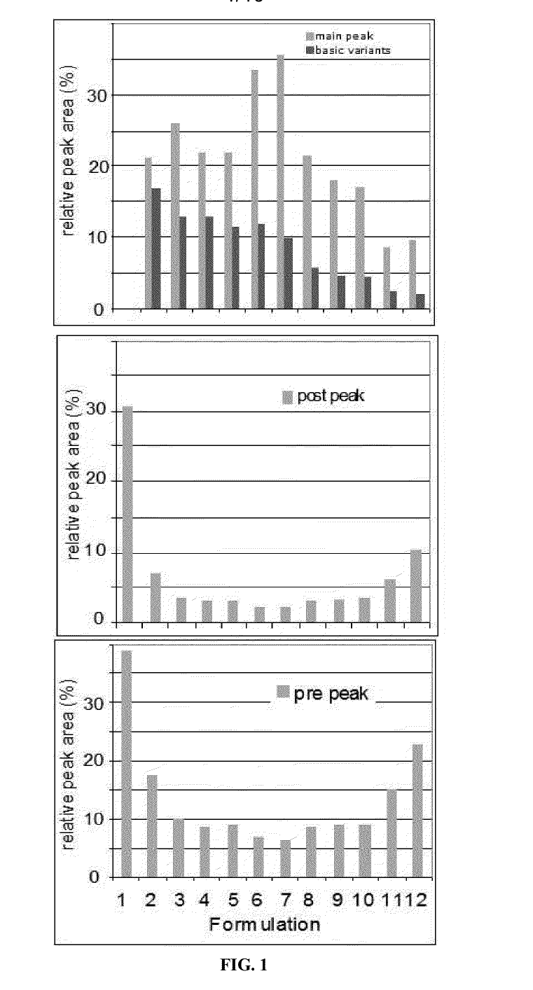

Basic peak area varied as a function of pH. The relative amount of aTSLP basic variants was the highest in the most acidic conditions (increased to 16.7% in citrate pH 4.0 [B2]; 14.8% at the initial time point), sensibly decreased in formulations ranging from citrate pH 5.0 [B4] to histidine pH 6.0 (at about 10%, [B8]) and further reduced to 5% and below in formulations with pH ranging from 6.5 to 8.0 ([B9] to [B12]). Of all the conditions tested, aTSLP formulated in histidine buffer at pH 5.5 [B6] retained the highest relative area for the main peak (about 35%), one of the smallest decrease in basic variants, and by way of consequence the smallest increase in acidic variants. See FIG. 1.

The gross changes observed in the IEX-HPLC chromatogram were also evident in RP-HPLC chromatograms of aTSLP samples stored at elevated temperature for 13 weeks. Storage of aTSLP at 40° C. in all formulations resulted in the formation of species eluting sooner than the main antibody peak (pre-peak elution at 2.55 min vs 2.85 min) by RP-HPLC.

The most dramatic changes were observed for the samples stored in the most acidic and basic conditions. Storage of aTSLP in citrate pH 3.0 [B1] resulted in the formation of a RP-HPLC pre-peak representing more than 30% of the integrated peak area after 13 weeks. After 13 weeks, the RP-HPLC pre peak area was comprised between 15 and 25% of the overall integrated area for samples stored in citrate pH 4.0 [B2], phosphate pH 7.0 [B11] and Tris-HCl pH 8.0 [B12], and remained lower than 10% in acetate pH 4.0 [B3] to citrate pH 7.0 [B10] (with pH ranging from 4.0 to 6.5). The increase in pre peak area corresponded to the reduction in peak height and area of the main antibody peak. By RP-HPLC, the histidine formulation at pH 5.5 [B6] retained the highest relative area for the main peak (up to 91%) and one of the smallest increases in RP-HPLC pre-peak variants (increase to about 6.9%).

The increase of the early eluting peak by RP-HPLC correlated with the formation of a late eluting peak by SEC-HPLC (˜26 min vs 32 min for the post-peak) and the reduction of the main peak height. This was particularly striking for the aTSLP sample stored in citrate pH 3.0 [B1]. In this condition, after 13 weeks, the SEC-HPLC post-peak accounted for up to 30% of the integrated peak area. The same phenomenon, albeit to a lesser extent, was also noticed for aTSLP formulated in citrate pH 4.0 [B2], phosphate pH 7.0 [B10] and in Tris-HCl pH 8.0 [B12]; the post-peak representing between 5 and 10% of the overall peak area. In citrate pH 4.0 [B3] to citrate pH 7.0 [B10], the post peak remained well below 5% of the overall integrated peak area. By SEC-HPLC, the histidine formulation at pH 5.5 retained the highest relative area for the main peak (97.4%) and the smallest increase in post-peak variants (increase to about 2.2%).

A late eluting peak by SEC-HPLC is indicative of the formation of a species with molecular weight lower than that of a monoclonal antibody monomer. Calibration of the S200 SEC-HPLC column with molecular markers indicated a retention time of the aTSLP fragment consistent with that of a globular protein of 25-30 kDa. Analysis by reducing SDS page of the 12 aTSLP formulations stored after 4 weeks at 40° C. revealed that the smaller molecular weight component likely come from the degradation of the heavy chain. Indeed, samples exposed to the most acidic or basic conditions presented, in addition to the expected heavy and light chains, bands at about 30-32 kDa absent from the samples stored in less stringent conditions and/or lower temperatures.

Aggregation, usually manifested by an increase of peak(s) eluting sooner than the main antibody peak by SEC-HPLC, remained relatively low during the 13 weeks of the study for aTSLP. Considering the unusual hydrophobicity of the molecule, this result was somewhat surprising. Since very large aggregates could potentially be filtered away on the SEC-HPLC column we analyzed the samples by DLS. No aggregates were found at these conditions.

Comparison of Histidine pH 5.5, Citrate pH 5.0 and Acetate pH 5.0 aTSLP Formulations:

In order to select the best potential formulation buffer from the three best conditions we compared the evolution as a function of time of the main peaks by IEX-, SEC- and RP-HPLC as well as basic peaks, post-peak and pre peak by IEX-, SEC- and RP-HPLC respectively.

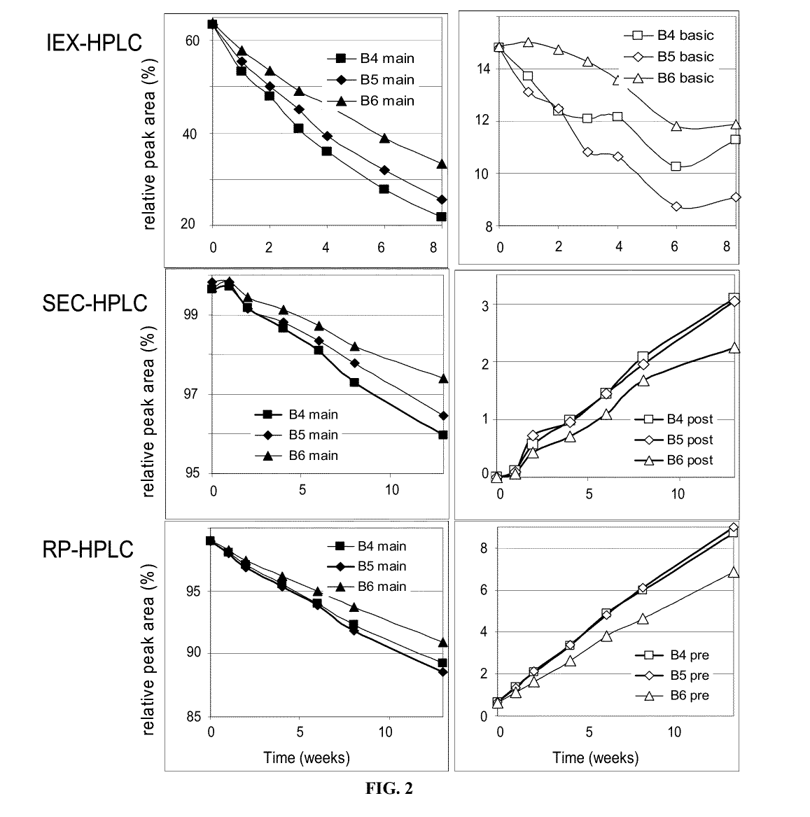

The three analytical methods independently indicated that the main peak retained its highest area when aTSLP was formulated in histidine pH 5.5 [B6]. As judged by the area of the main peak in IEX- and SEC-HPLC, the second best formulation was acetate pH 5.0 [B5]. However, the main peak area evolution by RP-HPLC was comparable in citrate and acetate pH 5.0 [B4 and B5]. The evolution of aTSLP basic variant by IEX-HPLC revealed the same order for preferential formulation conditions: lowest decrease was observed with histidine pH 5.5 [B6] followed by acetate pH 5.0 [B5] and citrate pH 5.0 [B4]. When considering evolution of the RP-HPLC pre peak and SEC-HPLC post peak, histidine pH 5.5 [B6] was again the formulation of choice since the increase of these variants was slower than in the two other formulations. Evolution of RP-HPLC pre peak and SEC-HPLC post peak for aTSLP in acetate and citrate pH 5.0 [B4 & B5] was almost identical. See FIG. 2.

TABLE 2
Relative peak area (%) by IEX-, SEC- and RP-HPLC after 8 weeks
(IEX-) and 13 weeks (SEC- and RP-HPLC) at 40° C.

| | IEX-HPLC | | SEC-HPLC | | RP-HPLC | |
|---|---|---|---|---|---|---|
| | main | basic | main | post | main | pre |
| | peak | variant | peak | peak | peak | peak |
| citrate pH 5.0 | 21.8% | 11.3% | 96.0% | 3.0% | 89.2% | 8.7% |
| [B4] | | | | | | |
| acetate pH 5.0 | 25.6% | 9.1% | 96.4% | 3.0% | 88.6% | 9.0% |
| [B5] | | | | | | |
| histidine pH 5.5 | 33.3% | 11.9% | 97.4% | 2.2% | 90.9% | 6.9% |
| [B6] | | | | | | |

### Stability at 25° C.

aTSLP formulated in the 12 different solutions was analyzed at 2, 4, 6, 8 and 13 weeks by IEX-HPLC, SEC-HPLC and RP-HPLC.

Comparison of all 12 Formulations Conditions:

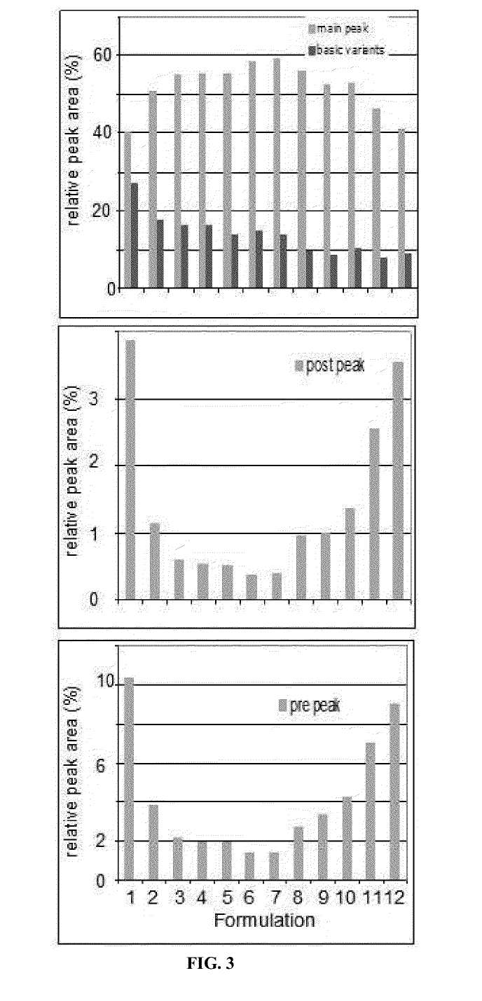

The modifications in the IEX-, SEC- and RP-HPLC chromatograms for the samples stored at 25° C. for up to 13 weeks were not as drastic as when they were subjected to 40° C. storage. See FIG. 3. However, changes were significant enough to confirm the trends we observed for the aTSLP samples stored at 40° C.; these include (i) formation of acidic variants, decrease in main peak and basic peak area by IEX HPLC (ii) formation of post peak by SEC-HPLC and (iii) formation of a pre peak by RP-HPLC.

As for the samples stored at 40° C., the most drastic changes in IEX-, SEC- and RP-HPLC chromatograms occurred for the formulations sitting at both extremities of the pH range. By IEX-HPLC, samples stored in Tris-HCl pH 8.0 [B12] and citrate pH 3.0 [B1] had the most severe reduction in main peak height and area (down to about 40% of the integrated area). As seen earlier, the decrease in main peak area was compensated by an increase in acidic peak area. The impact of pH on the basic peak area seen at 40° C. was also confirmed: basic peak area increased in the most acidic conditions (to about 25% in citrate pH 3.0 [B1]) while being severely reduced in the most basic conditions (less than 10% in Tris-HCl pH 8.0 [B12]). By SEC-HPLC, the largest increase of post peak was observed for citrate pH 3.0 [B1] (close to 4%) and Tris-HCl pH 8.0 ([B12] about 3.5%) with a minimum at a pH ranging from pH 5.0 to pH 6.0 (2.2% in histidine pH 5.5 [B6]). By RP-HPLC, the largest increase in pre peak relative area was again in citrate pH 3.0 [B1] (to above 10%) and Tris-HCl pH 8.0 [B12] (to about 9%), with again a minimum for formulations with a pH ranging from 5.0 to 6.0 (1.45% for histidine pH 5.5 [B6]), thus following exactly the trend observed for samples stored at 40° C.

In a manner consistent to what we observed for samples placed at 40° C., citrate pH 5.0 [B4], acetate pH 5.0 [B5] and histidine pH 5.5 [B6] were the best formulations since aTSLP formulated in these buffers retained acceptable main peak and basic peak area by IEX-HPLC while minimizing the formation of pre peak and post peak by RP-HPLC and SEC-HPLC, respectively.

Comparison of Histidine pH 5.5, Citrate pH 5.0 and Acetate pH 5.0 aTSLP Formulations:

We compared directly the stability data obtained at 25° C. in the three best buffers (citrate pH 5.0 [B4], acetate pH 5.0 [B5] and histidine pH 6.0 [B6]). We compared as earlier, the evolution of the main peaks as a function of time by IEX-, SEC- and RP-HPLC as well as basic peaks, post-peak and pre peak by IEX-, SEC- and RP-HPLC, respectively.

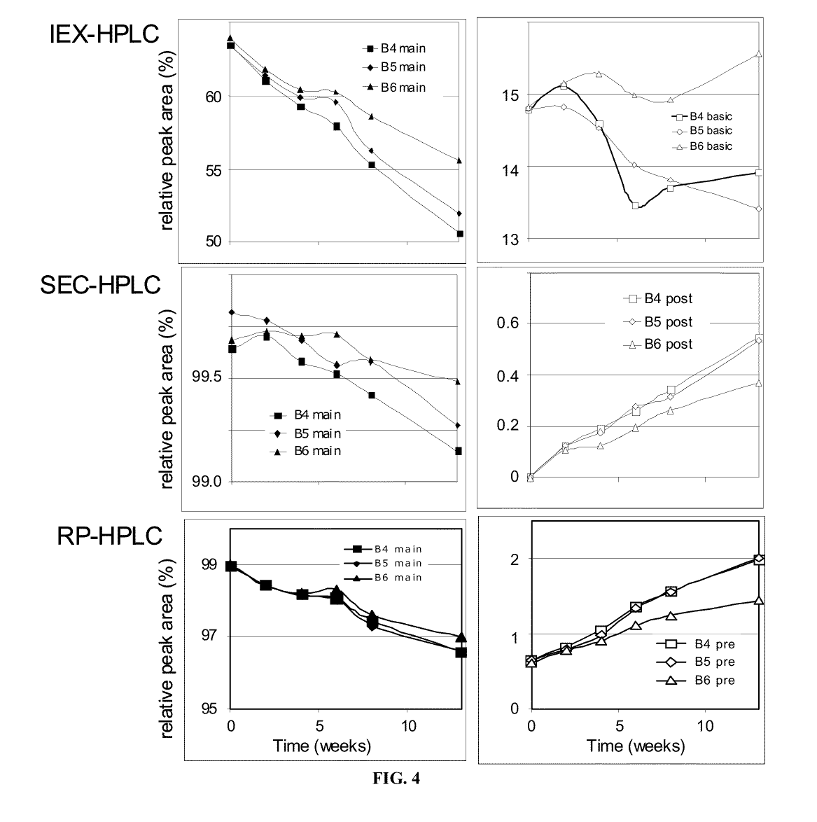

Again, the three analytical methods independently indicated that the main peak retained its highest area when aTSLP was formulated in histidine pH 5.5 [B6]. As judged by the area of the main peak in IEX- and SEC-HPLC, the second best formulation at 25° C. was again acetate pH 5.0 [B5]. Main peak area evolution by RP-HPLC was comparable in citrate and acetate pH 5.0 [B4 and B5] as seen at 40° C. The histidine buffer at pH 5.5 [B6] showed the lowest decrease of aTSLP basic variant by IEX-HPLC and hence is the preferred formulation (See FIG. 4 and Table 3). In contrast to the 40° C. samples, the second best formulation was citrate pH 5.0 [B4] followed by acetate pH 5.0 [B5]. When considering evolution of the RP-HPLC pre peak and SEC-HPLC post peak, histidine pH 5.5 [B6] was again the formulation of choice since the increase of these variants was slower than in the two other formulations. Evolution of the RP-HPLC pre peak and SEC-HPLC post peak for aTSLP in acetate and citrate pH 5.0 [B4 & B5] were identical.

TABLE 3
Relative peak area (%) by IEX-, SEC- and
RP-HPLC after 13 weeks at 40° C.

| | IEX-HPLC | | SEC-HPLC | | RP-HPLC | |
|---|---|---|---|---|---|---|
| | main | basic | main | post | main | pre |
| | peak | variant | peak | peak | peak | peak |
| citrate pH 5.0 | 50.6% | 13.9% | 99.1% | 0.5% | 96.6% | 2.0% |
| [B4] | | | | | | |
| acetate pH 5.0 | 52.0% | 13.4% | 99.3% | 0.5% | 96.6% | 2.0% |
| [B5] | | | | | | |
| histidine pH 5.5 | 55.6% | 15.6% | 99.5% | 0.4% | 97.0% | 1.5% |
| [B6] | | | | | | |

### Stability at 4° C.

aTSLP formulated in the 12 different solutions at 4° C. was analyzed at 4, 6 weeks and 13 weeks by IEX-HPLC, SEC-HPLC and RP-HPLC.

Comparison of all 12 Formulations Conditions:

The modifications in the IEX-, SEC- and RP-HPLC chromatograms for the samples stored at 4° C. for up to 13 weeks were minimum. Particularly, no significant trend was found in IEX chromatograms: the main peak height and area remained stable across the pH range, and the basic peak area only moderately varied in the different conditions. However, some changes in the most extreme pH conditions were noted by SEC-HPLC and RP-HPLC: formation of a post peak and pre peak, respectively. With the two latter analytical methods the general trend observed at 40° C. and 25° C. was also observed for samples stored at 4° C.

By SEC-HPLC, the larger increase of post peak was observed for Tris-HCl pH 8.0 (0.38%) and citrate pH 3.0 [B1] (close to 0.2%) with a minimum at pH ranging from pH 5.0 to pH 6.0 (0.05-0.06%). By RP-HPLC, the larger increase in pre peak relative area was in Tris-HCl pH 8.0 [B12] (to 1.3%) and in citrate pH 3.0[B1] (to about 1%) with a minimum for formulations with pH ranging from 5.0 to 6.0 (to 0.5%-0.6%).

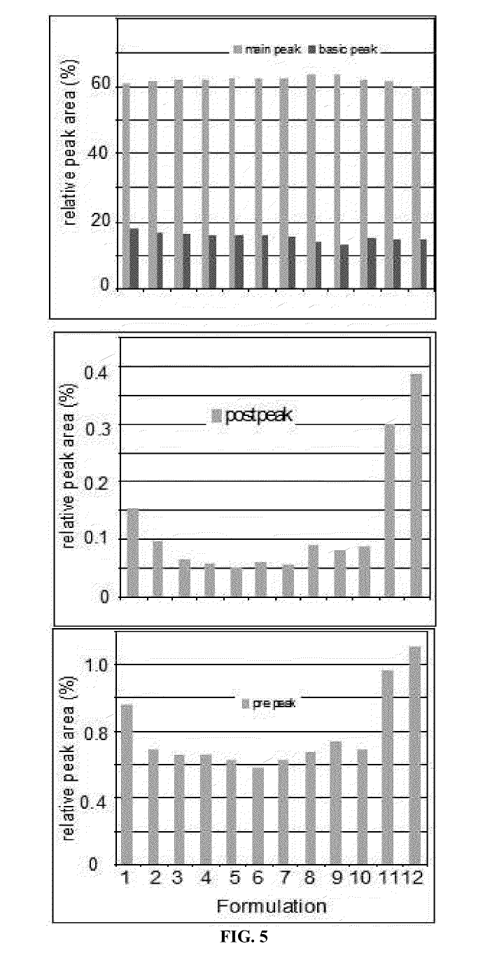

In a manner consistent to what we observed for samples placed at 40° C. and 25° C., citrate pH 5.0 [B4], acetate pH 5.0 [B5] and histidine pH 5.5 [B6] were the best formulations since aTSLP formulated in these buffers retained acceptable main peak and basic peak area by IEX-HPLC while minimizing the formation of pre peak and post peak by RP-HPLC and SEC-HPLC respectively. See FIG. 5.

Comparison of Stability Data at 40° C. And 25° C. For Histidine Buffer at pH 5.5 and pH 6.0:

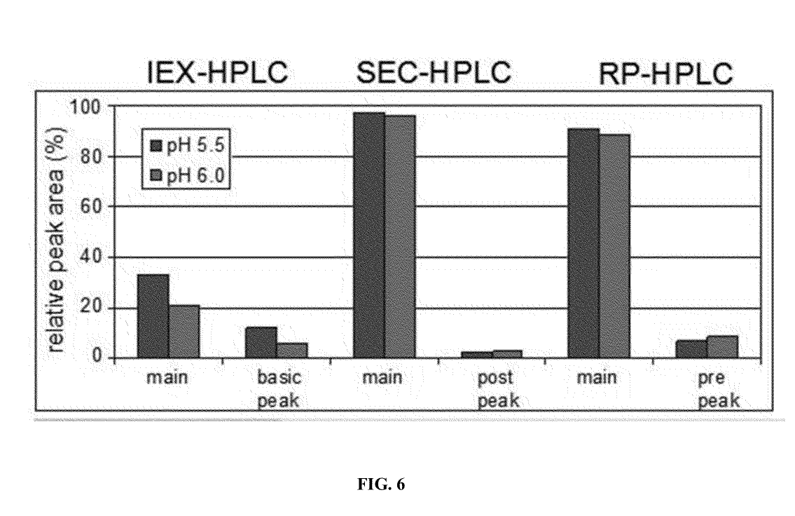

Our preformulation studies indicate that histidine pH 5.5 is the preferred formulation. At this stage, to have an initial idea of the pH range, we compared the stability studies generated in histidine pH 5.5 [B6] and histidine pH 6.0 [B8] at 40° C. and 25° C. A 0.5 pH unit variation resulted in changes in the chromatographic profiles; most significantly for IEX-HPLC. See FIG. 6. After 8 weeks at 40° C., the IEX-HPLC main peak went from 33.3% in histidine pH 5.5 to 21.3% at pH 6.0. The basic peak followed the same trend since an increase by 0.5 pH value resulted in a decrease of the basic peak of about 6 percentage points (from 11.8% to 5.6%).

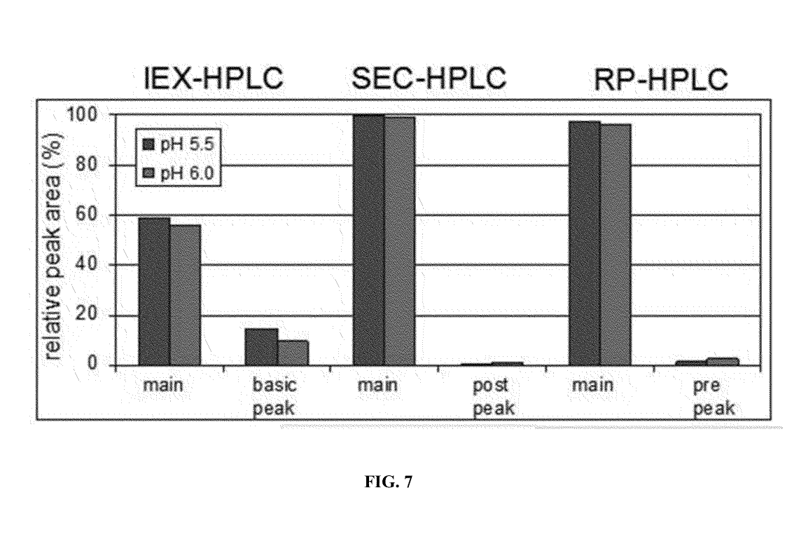

The same trend was also observed for stability studies performed with samples stored at 25° C. for 13 weeks. See FIG. 7. The most significant changes were observed on the IEX-HPLC chromatograms. An increase of pH from 5.5 to 6.0 resulted in the decrease of the main peak area from 58.6% to 55.9% and a decrease of the basic variants peak area by 5.3 percentage point (from 14.9% to 9.6%). Variations were also observed by SEC-HPLC and RP-HPLC both at 40° C. and 25° C., but remained limited.

### Discussion and Conclusion

Monoclonal antibodies are large and complex molecules subjected to a number of well identified degradation routes upon storage for extended periods of time. Such degradation typically includes, aggregation, heavy chain or light chain proteolitic cleavage, oxidation, deamidation, isomerisation etc. Determination of an optimal formulation for the Drug Substance is therefore of chief importance as it will control and reduce the occurrence of the aforementioned degradation products. Our preformulation studies performed over a large range of pHs and buffer species for up to 13 weeks at three staging temperatures (4° C., 25° C. and 40° C.) identified 20 mM histidine pH 5.5 as our lead formulation. In these conditions the SEC-HPLC post peak and RP-HPLC prepeak were at their minimum, while the IEX-HPLC main peak and basic peak remained acceptably high.

Increasing the histidine buffer pH by 0.5 pH unit to pH 6.0 had a detectable impact on the IEX-HPLC chromatogram, suggesting that the recommended pH range should be rather narrow. The pH of histidine solution, like many other standard buffers, is known to have a strong dependency on temperature (0.02 pH unit/° C.).

### REFERENCES

Gaza-Bulseco, G., Faldu, S., Hurkmans, K., Chumsae, C. and Liu, H. (2008) Effect of methionine oxidation of a recombinant monoclonal antibody on the binding affinity to protein A and protein G. J. Chrom. B 870, 55-62.
Pan, H., Chen, K., Chu, L., Kinderman, F., Apostol I. and Huang, G. (2009) Methionine oxidation in human IgG2 Fc decreases binding affinities to protein A and FcRn. Prot. Sci. 18, 424-433.
Wang, W., Singh, S., Zeng, D.L., King K. And Nema S. (2007) Antibody structure, Instability, and formulation. J. Pharm. Sci. 96, 1-26
Cordoba, A., Shyong, B.-J., Breen, D. and Harris, R. J. (2005) Non-enzymatic hinge region fragmentation of antibodies in solution. J. Chrom. B 818, 115-121.

### Example 3

### Toxicological Studies

This Example describes the development of a toxicology formulation for anti-TSLP (aTSLP). Protein aggregation is the major degradation pathway during the freeze-thaw process as well as during product handling and shipping. In order to determine the most robust formulation, stressing via freeze-thaw as well as shaking studies were carried out. Analytical size exclusion chromatography as well as turbidity by absorbance at 320 nm were applied to monitor product aggregation and degradation.

Based on the preformulation studies described in Example 2, three different buffer components (10 mM histidine, pH 5.5, 20 mM acetate, pH 5.3 and 10 mM citrate, pH 5.3) were chosen to be further tested with and without excipients for their suitability as toxicology formulation for anti-TSLP. Toxicology formulations are provided as frozen material (<−70° C.).

40.5 mg/ml of anti-TSLP (having the sequence described in Example 1) was tested in these buffers with and without excipients (7% sucrose, 0.02% polysorbate 80) for degradation and aggregation upon freeze-thaw and shaking stress. In particular, 0, 1, 2, 5-10 cycles of freeze-thaw cycles and shaking studies at 300 rpm over 3 days were undertaken in order to determine the most robust formulation. Size exclusion chromatography and turbidity were used as methods to evaluate product degradation and aggregation. anti-TSLP in 10 mM histidine, 7% sucrose, 0.02% polysorbate 80, pH 5.5 showed the most resistance towards freeze-thaw and shaking stress.

### Materials

The following stock solutions were prepared:

Solution A=200 mM acetate, pH 5.3

Solution B=100 mM histidine, pH 5.5

Solution C=100 mM citrate, pH 5.3

Solution D=5% polysorbate 80

Solution E=50% sucrose

Solution A-solution E were mixed in different ratios to obtain:

Buffer A0=20 mM acetate, pH 5.3

Buffer A1=20 mM acetate, 7% sucrose, pH 5.3

Buffer A2=20 mM acetate, 0.02% polysorbate 80, pH 5.3

Buffer A3=20 mM acetate, 7% sucrose, 0.02% polysorbate 80, pH 5.3

Buffer H0=10 mM histidine, pH 5.5

Buffer H1=10 mM histidine, 7% sucrose, pH 5.5

Buffer H2=10 mM histidine, 0.02% polysorbate 80, pH 5.5

Buffer H3=10 mM histidine, 7% sucrose, 0.02% polysorbate 80, pH 5.5

Buffer C0=10 mM citrate, pH 5.3

Buffer C1=10 mM citrate, 7% sucrose, pH 5.3

Buffer C2=10 mM citrate, 0.02% polysorbate 80, pH 5.3

Buffer C3=10 mM citrate, 7% sucrose, 0.02% polysorbate 80, pH 5.3

anti-TSLP was made according to Example 1:

52.4 mg/ml anti-TSLP in 10 mM citrate, pH 5.3

49 mg/ml anti-TSLP in 10 mM histidine, pH 5.5

473 mg/ml anti-TSLP in 20 mM acetate, pH 5.3

Stock solutions A-E were added to the respective anti-TSLP solution to obtain 5 ml of 40.5 mg/ml anti-TSLP in buffer A0, A1, A2, A3, C0, C1, C2, C3, H0, H1, H2, H3 (batch 67683-83). These solutions were used for further freeze-thaw and shaking.

### Methods

Freeze-Thaw Cycles:

For all buffer conditions 5 vials (2 ml, Corning 430915) were prepared to have one vial for each freeze-thaw cycle (cycle 0, 1, 2, 5 and 10), filled with 500 μl of solution. For cycle 0 the solutions were left in the refrigerator. For each freezing cycle, the solutions were put into the <−70° C. freezer for at least one hour. Thawing time was at least 45 minutes. All cycles were finished at the same time. Unused samples were frozen at −70° C. for possible future analysis.

Shaking Setup:

500 μl of each anti-TSLP solution was aliquoted in a 2 ml polypropylene vial (Corning 430915). All vials were set up into a horizontal position on a shaker at ambient room temperature. The shaker was set to 300 rpm for 3 days.

Size Exclusion Chromatography:

Size exclusion chromatography was run on an Agilent HPLC system with a Superdex 10/300 column. Flow rate was 0.5 ml/min and run time 60 min. The injection volume was 10 μl for cycle 0 and cycle 5 and 75 μl for cycle 10. The samples were not diluted before the injection. A Biorad protein gel filtration marker (Cat.: 151-1901), including proteins with molecular weights ranging from 1.35 kDa to 670 kDa, was used as a standard to estimate the molecular weight of the eluting species.

Turbidity Studies:

The apparent absorbance at 320 nm was monitored in an Agilent spectrophotometer using 1 cm path length cells. The buffer absorbance was subtracted from the anti-TSLP absorbance to get the final readings.

### Results and Discussion

Freeze-Thaw Study:

Size exclusion chromatography and turbidity measurements were carried out after freeze-thaw cycle 0, 5 and 10. Turbidity studies were only done for the initial (cycle 0) and after cycle 10. A typical size exclusion chromatogram shows a main monomer peak, an aggregate peak (peak1), a trimer-tetramer peak (peak 2) and a small fraction of a degradation product (post peak). The molecular weight of these peaks are estimated based on the retention times by running a protein gel filtration standard (Table 4).

Tables 5-7 show the effect of buffer on the degradation and aggregation behavior of anti-TSLP for freeze-thaw cycle 0, 5 and 10, when analyzed by size exclusion chromatography.

By comparing aggregate levels in the initial samples (prepeak 1 and prepeak 2 in Table 2) it can be seen that anti-TSLP in all histidine buffers has the lowest levels of aggregates. In formulations without excipients and in formulations containing polysorbate 80 only anti-TSLP forms higher order aggregates as well as trimers-tetramers. Sucrose shows to prevent aggregation in all formulations up to freeze-thaw cycle 5.

Absorbance studies at 320 nm do not show any big increase comparing initial and freeze-thaw cycle 10 for all formulations, with the exception of citrate buffer (CO-buffer).

TABLE 4
Molecular weight estimates of anti-TSLP from size exclusion
chromatography (batch 67683-148)

| Prepeak 1 | Prepeak 2 | Main peak | Post peak |
|---|---|---|---|
| 1789 kDa | 565 kDa | 169 kDa | 25 kDa |

TABLE 5
Dependence of buffer on degradation and aggregation of
anti-TSLP species upon freeze-thaw analyzed by size
exclusion chromatography-cycle 0 (batch 67683-95)

| Buffer | Prepeak 1 (%) | Prepeak 2 (%) | Main peak (%) | Postpeak (%) |
|---|---|---|---|---|
| A0 | 0.23 | 0.11 | 99.64 | 0.02 |
| A1 | 0.24 | 0.10 | 99.63 | 0.02 |
| A2 | 0.23 | 0.11 | 99.64 | 0.02 |
| A3 | 0.21 | 0.10 | 99.67 | 0.02 |
| C0 | 0.06 | 0.11 | 99.80 | 0.02 |
| C1 | 0.07 | 0.10 | 99.80 | 0.03 |
| C2 | 0.06 | 0.11 | 99.81 | 0.02 |
| C3 | 0.06 | 0.10 | 99.82 | 0.02 |
| H0 | 0.08 | 0.07 | 99.84 | 0.02 |
| H1 | 0.07 | 0.06 | 99.85 | 0.02 |
| H2 | 0.06 | 0.07 | 99.86 | 0.02 |
| H3 | 0.05 | 0.06 | 99.86 | 0.02 |

Buffer notation: A = 20 mM acetate, pH 5.3, C = 10 mM citrate, pH 5.3, H = 10 mM histidine, pH 5.5, 0 = no excipients added, 1 = 7% sucrose added, 2 = 0.02% polysorbate 80 added, 3 = 7% sucrose, 0.02% polysorbate 80 added.

TABLE 6
Dependence of buffer on degradation and aggregation of
anti-TSLP species upon freeze-thaw analyzed by size
exclusion chromatography-cycle 5 (batch 67683-95)

| Buffer | Prepeak 1 (%) | Prepeak 2 (%) | Main peak (%) | Postpeak (%) |
|---|---|---|---|---|
| A0 | 0.31 | 0.19 | 99.49 | 0.01 |
| A1 | 0.28 | 0.08 | 99.60 | 0.04 |
| A2 | 0.17 | 0.16 | 99.65 | 0.02 |
| A3 | 0.21 | 0.09 | 99.67 | 0.02 |
| C0 | 0.10 | 0.26 | 99.62 | 0.02 |
| C1 | 0.09 | 0.10 | 99.79 | 0.02 |
| C2 | 0.06 | 0.22 | 99.69 | 0.02 |
| C3 | 0.06 | 0.09 | 99.83 | 0.02 |
| H0 | 0.11 | 0.24 | 99.63 | 0.02 |
| H1 | 0.08 | 0.07 | 99.83 | 0.02 |
| H2 | 0.05 | 0.22 | 99.71 | 0.02 |
| H3 | 0.05 | 0.06 | 99.87 | 0.02 |

Buffer notation: A = 20 mM acetate, pH 5.3, C = 10 mM citrate, pH 5.3, H = 10 mM histidine, pH 5.5, 0 = no excipients and 3 = 7% sucrose, 0.02% polysorbate 80 added.

TABLE 7
Dependence of buffer on degradation and aggregation of
anti-TSLP species upon freeze-thaw analyzed by size
exclusion chromatography-cycle 10 (batch 67683-97).

| Buffer | Prepeak 1 (%) | Prepeak 2 (%) | Main peak (%) | Postpeak (%) |
|---|---|---|---|---|
| A0 | 0.33 | 0.37 | 99.30 | 0.00 |
| A1 | 0.28 | 0.12 | 99.54 | 0.06 |
| A2 | 0.20 | 0.41 | 99.34 | 0.04 |
| A3 | 0.23 | 0.13 | 99.61 | 0.03 |
| C0 | 0.19 | 0.44 | 99.35 | 0.02 |
| C1 | 0.31 | 0.17 | 99.50 | 0.02 |
| C2 | 0.10 | 0.12 | 99.76 | 0.03 |
| C3 | 0.13 | 0.39 | 99.46 | 0.01 |
| H0 | 0.22 | 0.41 | 99.34 | 0.03 |
| H1 | 0.13 | 0.08 | 99.76 | 0.02 |
| H2 | 0.12 | 0.39 | 99.47 | 0.02 |
| H3 | 0.19 | 0.10 | 99.68 | 0.03 |

Buffer notation: A = 20 mM acetate, pH 5.3, C = 10 mM citrate, pH 5.3, H = 10 mM histidine, pH 5.5, 0 = no excipients added, 1 = 7% sucrose added, 2 = 0.02% polysorbate 80 added, 3 = 7% sucrose, 0.02% polysorbate 80 added.

Shaking Study:

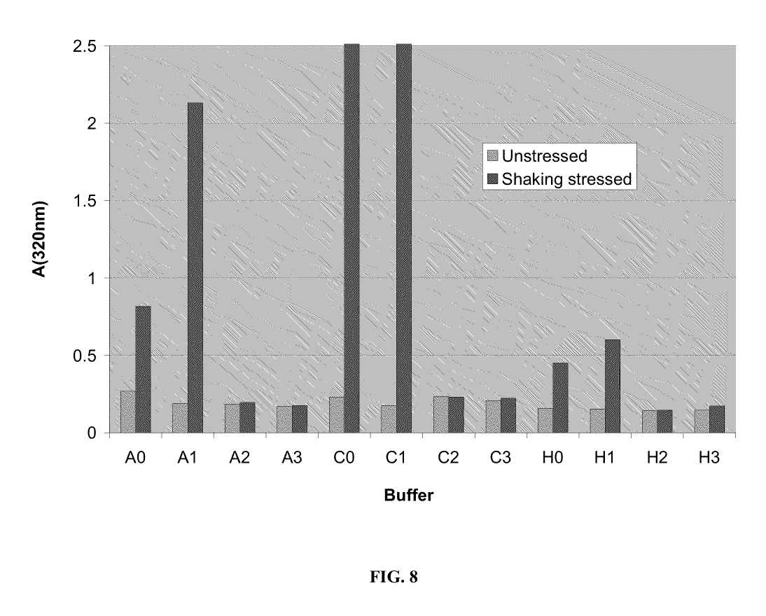

40.5 mg/ml anti-TSLP in all 12 formulations was stressed by shaking at 300 rpm at room temperature for 3 days and then analyzed by turbidity. Results are shown in FIG. 8. All formulations containing polysorbate 80 (A2, A3, C2, C4, H2, H3) show no sign of aggregation after being stressed by shaking. Among the formulations, which do not contain polysorbate 80, histidine formulations are found to be more resistant to aggregation than acetate and citrate formulations. anti-TSLP is shown to be prone to aggregation in citrate formulations, when no excipients are added.

### Conclusions

The freeze-thaw studies show that from all anti-TSLP formulations tested, all formulation containing sucrose (A1, A3, C1, C3, H1, H3) are resistant to aggregation up to freeze-thaw cycle 5. Acetate formulations seem to have a slightly increased level of aggregates initially compared to histidine formulations up to cycle 5 according to size exclusion data.

The shaking studies indicate that anti-TSLP formulations containing polysorbate 80 are resistant to shaking stress. Without excipients anti-TSLP in histidine formulations are less prone to aggregation than acetate formulation and citrate formulations during shaking stress.

### Example 4

### Preparation of Lyophilized Formulations for Subcutaneous and Intravenous Administration

The anti-TSLP antibody is formulated as a lyophilized powder which is reconstituted with sterile water for injection prior to use. The formulation was designed to be isotonic.

The lyophilization process was optimized taking into consideration moisture content, cake appearance and reconstitution time. Various biochemical and biophysical tests showed comparability of the lyophilized material with the pre-lyo solution.

The lyophilized formulation to be used for reconstitution to 40 mg/mL for intravenous injection consists of approximately 40 mg/mL aTSLP in 10 mM Histidine buffer, 7% Sucrose, 0.02% Polysorbate 80, pH 5.5. aTSLP Powder for Injection, 100 mg/vial, product is stored at 2-8° C. For a 40 mg/mL solution, the lyophilized product (Table 8) is reconstituted with 2.7 mL sterile water for injection to provide 40 mg/mL solution of aTSLP in 10 mM Histidine buffer, 7% (w/v) Sucrose, 0.02% (w/v) Polysorbate 80 and pH 5.5. aTSLP Powder for Injection, 100 mg/vial, product is stored at 2-8° C.

TABLE 8
Composition of aTSLP Powder for Injection, 100 mg/vial
(40 mg/mL aTSLP after reconstitution)

| | | Concentration | |
|---|---|---|---|
| | | after | |
| | Amount | Reconstitution | |
| Ingredient | (mg/vial)^{a} | (mg/mL)^{c} | Function |
| aTSLP | 100 | 40 | Active |
| | | | Pharmaceutical |
| | | | Ingredient |
| L-Histidine USP | 0.7195 | 0.2878 | Buffer |
| L-Histidine | 4.27 | 1.708 | Buffer |
| Monohydrochloride | | | |
| Monohydrate Ph.Eur/ | | | |
| B.P. | | | |
| Sucrose NF | 175 | 70 | Stabilizer/ |
| | | | Tonicity |
| | | | Modifier |
| Polysorbate 80 NF | 0.5 | 0.2 | Surfactant |
| Water for Injection^{b} | 2.5 mL @ q.s. | — | Solvent |
| USP | | | |

^{a}An excess fill of 0.4 mL is provided to ensure the recovery of the label claim of 100 mg aTSLP per vial
^{b}Water removed by sublimation during lyophilization
^{c}After reconstitution with 2.7 mL of sterile water for injection

The lyophilized formulation to be used for reconstitution to 100 mg/mL for subcutaneous injection consists of 4 mM Histidine buffer, 2.9% Sucrose, 0.008% Polysorbate 80, pH 5.5. aTSLP Powder for Injection, 100 mg/vial, product is stored at 2-8° C. For each vial, 3.4 mL of above solution will be filled in to 10R DIN vials and lyophilized. Each lyophilized vial will be reconstituted with 1.2 mL of WFI prior to use to achieve 100 mg/mL aTSLP in 10 mM histidine, 7.25% sucrose, 0.02% polysorbate 80, pH 5.5. The reconstituted solution will be isotonic for SC injection.

For a 100 mg/mL solution, the lyophilized product (Table 9) is reconstituted with 1.2 mL sterile water for injection to provide 100 mg/mL solution of aTSLP in 10 mM Histidine buffer, 7.25% (w/v) Sucrose, 0.02% (w/v) Polysorbate 80 and pH 5.5.

TABLE 9
Composition of aTSLP Powder for Injection, 100 mg/vial
(100 mg/mL aTSLP after reconstitution)

| | | Concentration | |
|---|---|---|---|
| | | after | |
| | Amount | Reconstitution | |
| Ingredient | (mg/vial)^{a} | (mg/mL)^{c} | Function |
| aTSLP | 100 | 100 | Active |
| | | | Pharmaceutical |
| | | | Ingredient |
| L-Histidine USP | 0.2878 | 0.2878 | Buffer |
| L-Histidine | 1.708 | 1.708 | Buffer |
| Monohydrochloride | | | |
| Monohydrate Ph.Eur/ | | | |
| B.P. | | | |
| Sucrose NF | 72.5 | 72.5 | Stabilizer/ |
| | | | Tonicity |
| | | | Modifier |
| Polysorbate 80 NF | 0.2 | 0.2 | Surfactant |
| Water for Injection^{b} | 2.5 mL @ | — | Solvent |
| USP | q.s. | | |

^{a}An excess fill of 0.9 mL is provided to ensure the recovery of the label claim of 100 mg aTSLP per vial
^{b}Water removed by sublimation during lyophilization
^{c}After reconstitution with 1.2 mL of sterile water for injection

### Example 6

### Stability of Lyophilized aTSLP Formulations Stability Studies for Lyophilized aTSLP Formulation for Subcutaneous Injection

aTSLP Powder for Injection, 100 mg/vial is for subcutaneous injection and is packaged as a lyophilized drug product in 10 mL Type I tubing glass vial with a 13 mm lyo stopper and a 13 mm Flip-Off® seal. Recommended storage condition is 2° C. to 8° C. Single use lyophilized drug product in glass vial is reconstituted with sterile water for injection prior to use to achieve a concentration of 100 mg/mL at pH 5.5.

Research material (batch NB-liyun-0321710-0010) is staged on stability for up to 36 months. Clinical material (batch WL00046031) is staged on stability for up to 60 months. Currently, 6 months (batch NB-liyun-0321710-0010) and 1 month (batch WL00046031) of stability data is available. All results are within proposed specifications when stored at the recommended storage conditions of 2-8° C.

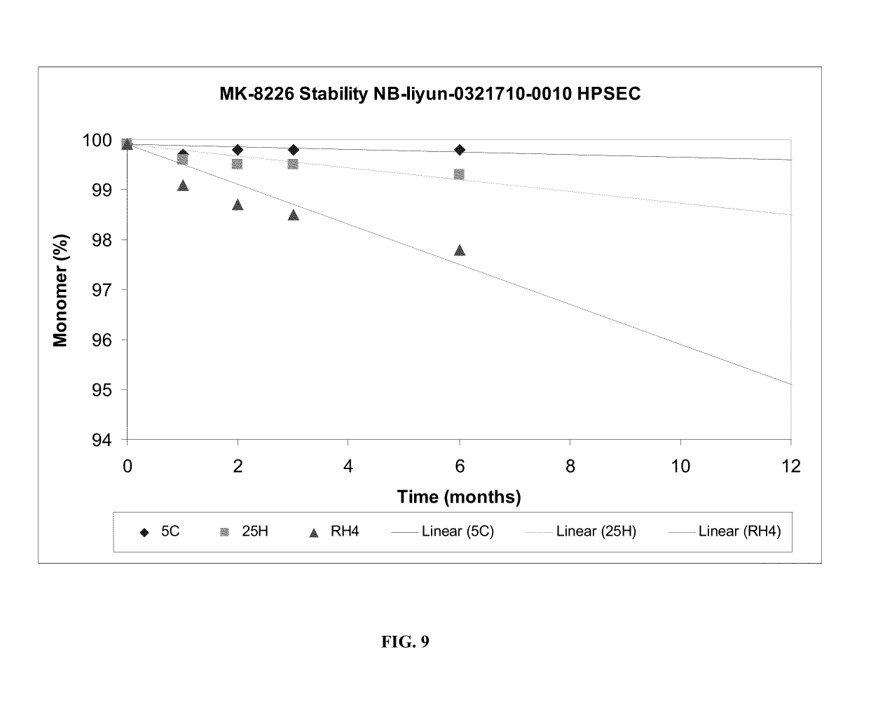

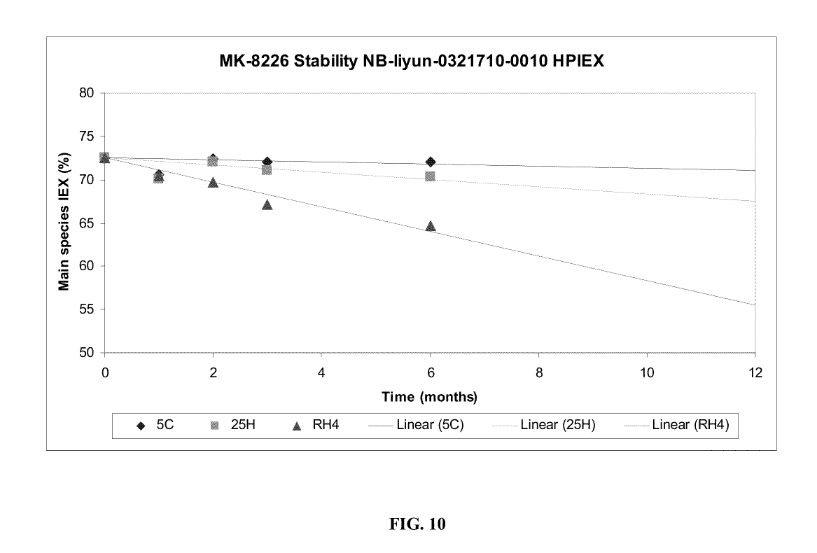

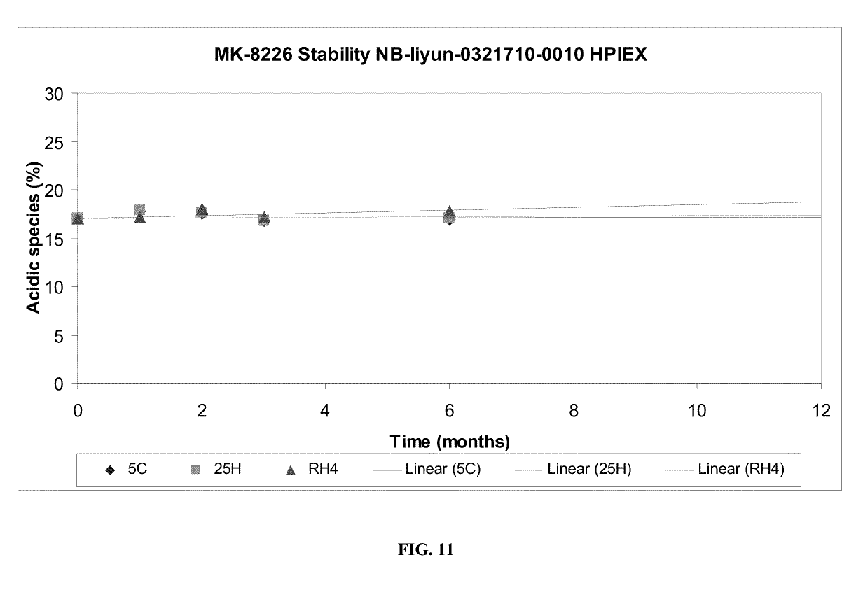

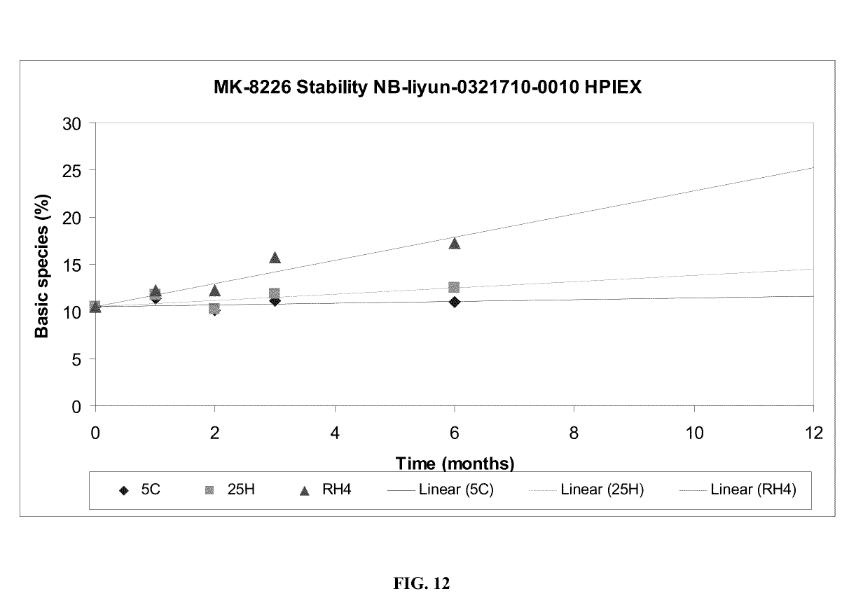

aTSLP Powder for Injection, 100 mg/vial research batch NB-liyun-0321710-0010 was manufactured on September 2011. A stability study was initiated on this batch to assess stability of proposed lyophilized formulation. Vials have been staged on stability at 5° C./ambient R.H. (5 C), 25° C./60% R.H. (25H) and 40° C./75% R.H. (RH4), with 2-8° C. being the recommended long term storage condition. The duration of this study is 36 months. Linear trends on the 6-month preclinical HP-SEC (% monomer) data shown in FIG. 9 indicate minimal changes for up to 12 months at 5° C. Based on these projections, samples at the recommended storage conditions of 2-8° C. are expected to meet their acceptance criteria of ≧95.0% monomer for at least 12 months. Linear trends on the 6-month HP-IEX data are provided in FIGS. 10, 11 and 12. These indicate minimal changes for up to 12 months at 5° C. At accelerated storage conditions of 25H and stress conditions of RH4, slight changes were observed in % moisture test results over six months. Moisture content showed an increase at both 25H condition from 0.6% to 0.8% and RH4 condition from 0.6% to 1.0%. HP-SEC percent monomer was found to reduce to 97.8% at RH4 condition and slightly reduced to 99.3% for 25H condition from the initial 99.9% with a corresponding increase in the HMW species. HP-IEX main species percentage was found to decrease to 70.3% and 64.7% at 25H and RH4 respectively from the initial 72.5% with a corresponding increase in the basic variants for both conditions and the acidic variants at RH4. Overall, the 6 months of preclinical stability data on lyophilized drug product shows no significant change to the product quality at refrigerated storage conditions and slight changes at the accelerated (25H) and stress (RH4) conditions.

aTSLP Powder for Injection, 100 mg/vial Phase I clinical drug product batch WL00046031 and WL00047366 were manufactured on December 2011 and March 2012, respectively. The single-use drug product is packaged in 10 mL Type I tubing glass vial stoppered with a 13 mm West 4432/50 lyo stopper and sealed with a 13 mm Flip-Off® seal. aTSLP Powder for Injection, 100 mg/vial is reconstituted with sterile water for injection prior to use. A stability study was initiated on batch WL00046031 in February 2012. Vials have been stored at 5 C, 25H and RH4, with 2-8° C. being the recommended long term storage condition. The duration of this study will be 60 months. The one month stability of Phase I clinical drug product batch WL00046031 has show no reduction of HP-SEC percent monomer or HP-IEX main species percentage in storage and accelerated stability conditions (5° C., 25H). At stressed condition RH4, HP-SEC percent monomer slightly reduced from 100.0% to 99.0% and HP-IEX main species percentage slightly reduced from 71.2% to 68.2%. This data showed stability consistency across different batches.

### Stability Studies for Lyophilized aTSLP Formulation for Intravenous Injection

aTSLP Powder for Injection, 100 mg/vial (FM005575-1-1) is for intravenous injection and is packaged as a lyophilized drug product in 10 mL Type I tubing glass vial with a 13 mm West 4432/50 lyo stopper and a 13 mm Flip-Off® seal. Intended storage condition is 2° C. to 8° C. Single use lyophilized drug product in glass vial is reconstituted with sterile water for injection prior to use to achieve a concentration of 40 mg/mL at pH 5.5.

Research material (batch 89782-101) is staged on stability for up to 24 months. Clinical material (batch WL00044338) is staged on stability for up to 60 months and clinical material from another batch (batch WL00044673) will be staged on stability for up to 60 month. Currently, 6 months (batch 89782-101) and 1 month (batch WL00044338) of stability data is available. All results are within proposed specifications when stored at the recommended storage conditions of 2-8° C. An initial shelf-life of 12 months has been proposed based on the available stability data.

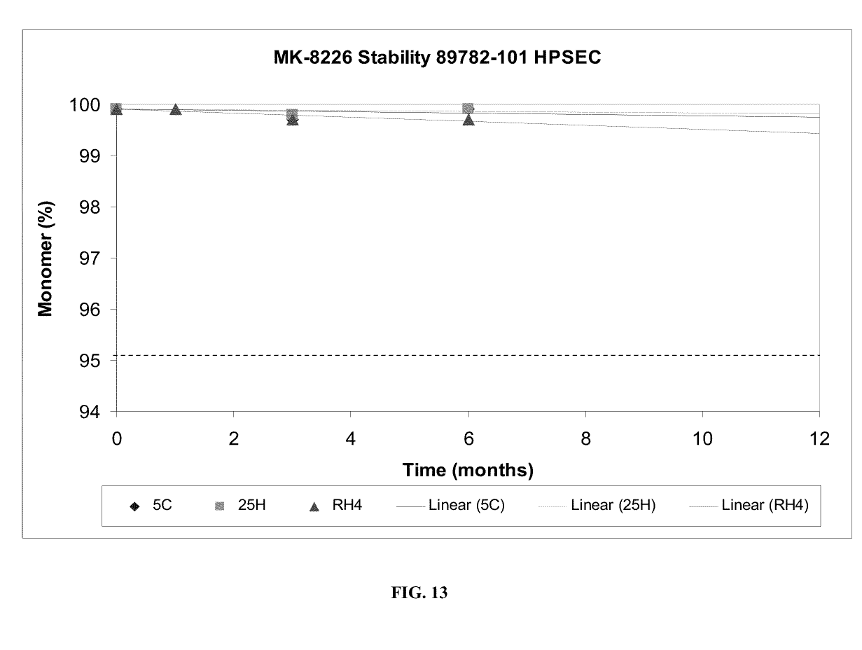

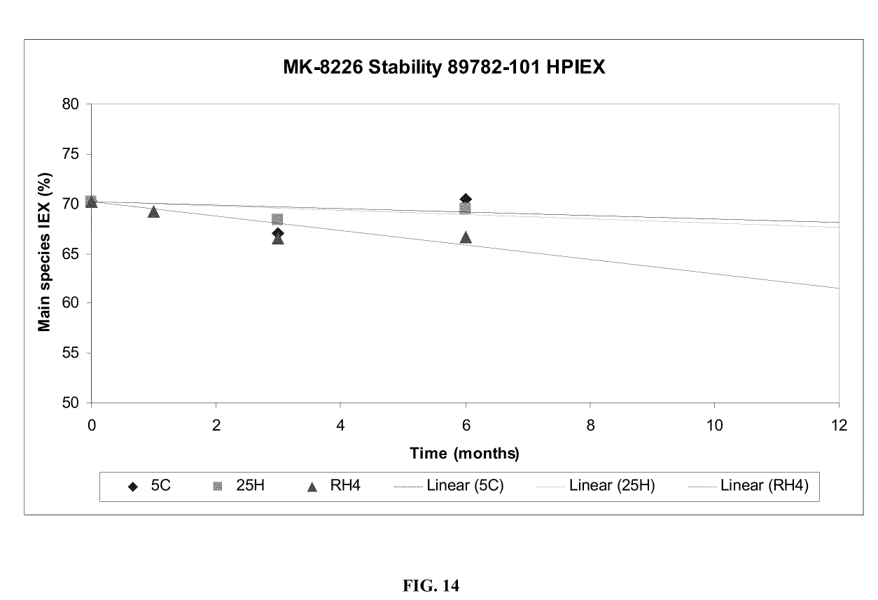

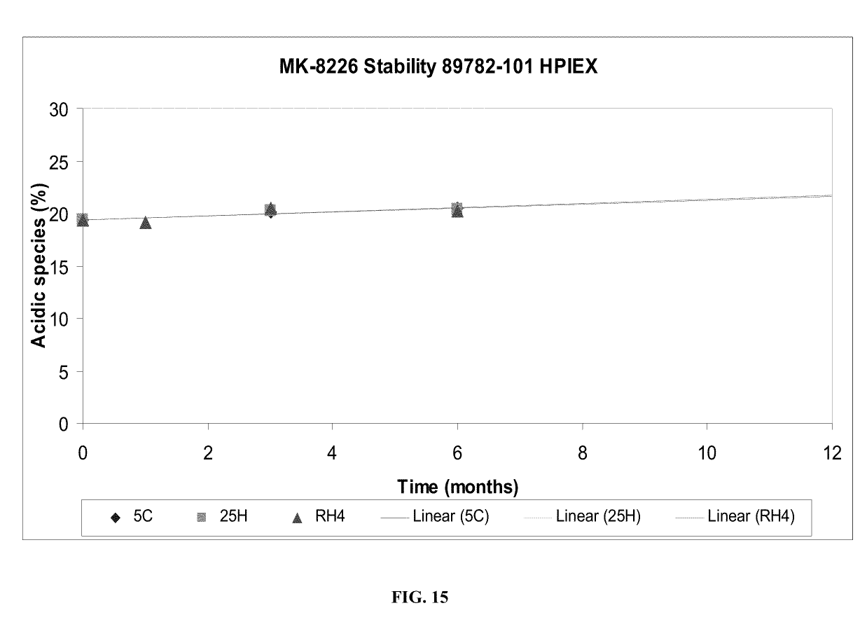

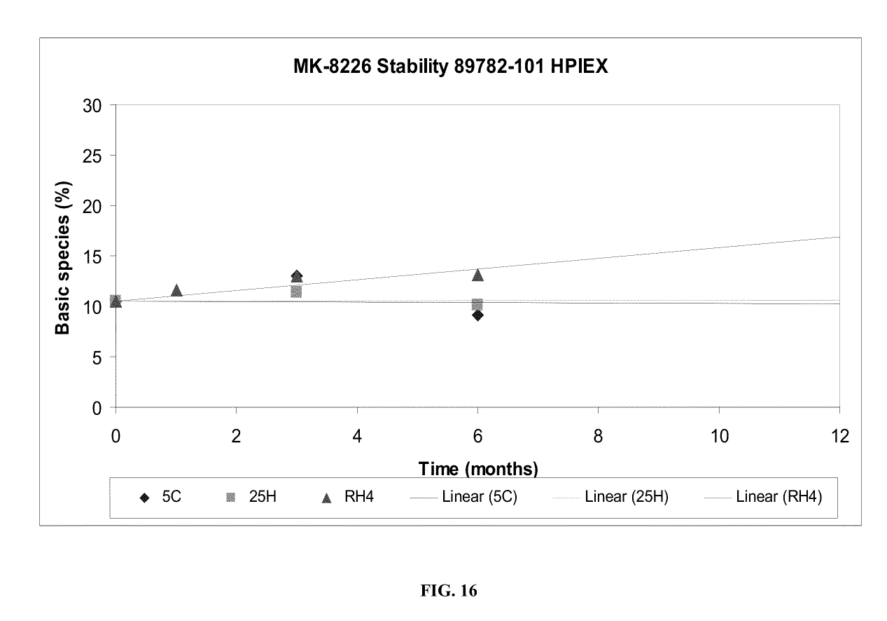

aTSLP Powder for Injection, 100 mg/vial research batch 89782-101 was subject to a study to assess stability of proposed lyophilized formulation. Vials have been staged on stability at 5° C./ambient R.H. (5 C), 25° C./60% R.H. (25H) and 40° C./75% R.H. (RH4), with 2-8° C. being the recommended long term storage condition. The duration of this study is 24 months. Linear trends on the 6-month HP-SEC (% monomer) data shown in FIG. 13 indicate minimal changes for up to 12 months at 5° C. Based on these projections, samples at the recommended storage conditions of 2-8° C. are expected to meet their acceptance criteria of ≧95.0% monomer for at least 12 months. Linear trends on the 6-month HP-IEX data are provided in FIGS. 14, 15 and 16. These indicate minimal changes for up to 12 months at 5° C. At accelerated storage conditions of 25H and stress conditions of RH4, slight changes were observed in % moisture test results over six months. Moisture content showed an increase at both 25H and RH4 conditions from 0.4% to 0.6%. HP-SEC percent monomer was found to slightly reduce to 99.7% at RH4 condition from the initial 99.9% with a corresponding increase in the HMW species and no reduction for 25H condition. HP-IEX main species percentage was found to decrease to 69.5% and 66.7% at 25H and RH4 respectively from the initial 70.2% with a corresponding increase in the acidic variants for both conditions and the basic variants at RH4. Overall, the 6 months of BSP stability data on lyophilized drug product shows no significant change to the product quality at refrigerated storage conditions and slight changes at the accelerated (25H) and stress (RH4) conditions.

aTSLP Powder for Injection, 100 mg/vial Phase I clinical drug product batch WL00044338 and WL00044673 were manufactured in West Point, Pa. (25 Aug. 2011 and 10 Nov. 2011 respectively) The single-use drug product is packaged in 10 mL Type I tubing glass vial stoppered with a 13 mm West 4432/50 lyo stopper and sealed with a 13 mm Flip-Off® seal. aTSLP Powder for Injection, 100 mg/vial is reconstituted with sterile water for injection prior to use. A stability study was initiated on batch WL00044338 in WAG (3 Nov. 2011). Vials have been stored at 5 C, 25H and RH4, with 2-8° C. being the recommended long term storage condition. The duration of this study will be 60 months. Another stability study will be initiated on batch WL00044673 in WAG (27 Jan. 2012). Vials will be stored at 5 C, 25H and RH4, with 2-8° C. being the recommended long term storage condition. The duration of this study will be 60 months. The one month formal stability of Phase I clinical drug product batch WL00044338 has show no reduction of HP-SEC percent monomer or HP-IEX main species percentage in all of the stability conditions (5° C., 25H and RH4). This data showed stability consistency across different batches.

### Example 7

### Viscosity of the aTSLP Formulations

The viscosity of the intravenous and subcutaneous formulations described above was measured by NimiVis II Viscometer (Grabner Instruments) at 20 C. The values obtained are summarized in Table 10.

TABLE 10

| | | Concentration | Viscosity |
|---|---|---|---|
| Formulation | Type of solution | (mg/mL) | (cP) |
| MK-8226 IV | Pre-lyo and post recon | 40 | 2.405 |
| Formulation | solution | | |
| MK-8226 SC | Pre-lyo solution | 40 | 1.845 |
| Formulation | Post recon solution | 100 | 3.939 |

As used herein, including the appended claims, the singular forms of words such as “a,” “an,” and “the,” include their corresponding plural references unless the context clearly dictates otherwise. Unless otherwise indicated, the proteins and subjects referred to herein are human proteins and subject, rather than another species.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. §1.57(b)(1), to relate to each and every individual publication, database entry (e.g. Genbank sequences or GeneID entries), patent application, or patent, each of which is clearly identified in compliance with 37 C.F.R. §1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

What is claimed is:

1. A formulation comprising an anti-TSLP antibody, or antigen binding fragment thereof and a histidine buffer.

2. The formulation of claim 1, further comprising a surfactant.

3. The formulation of claim 2, further comprising a stabilizer.

4. The formulation of claim 3, wherein the surfactant is polysorbate 80 and the stabilizer is sucrose.

5. The formulation of claim 4, wherein the formulation has a pH of about 5.5.

6. The formulation of claim 5, wherein the formulation is lyophilized.

7. The formulation of claim 6, wherein the formulation enables reconstitution of the anti-TSLP antibody, or antigen binding fragment thereof, at a concentration of between about 40 mg/mL and 100 mg/mL.

8. The formulation of claim 6, wherein after reconstitution the polysorbate 80 is present at a weight ratio of approximately 0.02% compared to the antibody or antigen binding fragment thereof.

9. The formulation of claim 6, wherein after reconstitution sucrose is present at a weight ratio of approximately 7% compared to the antibody, or antigen binding fragment thereof.

10. The formulation of claim 6, wherein after reconstitution the formulation comprises:
a) 40-100 mg/mL anti-TSLP antibody, or antigen binding fragment thereof;
b) about 70 mg/mL sucrose;
c) about 0.2 mg/mL polysorbate 80; and
d) about 10 mM Histidine buffer at about pH 5.0-pH 6.0.

11. The formulation of claim 6, made by lyophilizing an aqueous solution comprising:
a) 100 mg/mL anti-antibody, or antigen binding fragment thereof;
b) about 70 mg/mL sucrose;
c) about 0.2 mg/mL polysorbate 80; and
d) about 10 mM histidine buffer at pH 5.0-6.0.

12. The formulation of claim 6, made by lyophilizing an aqueous solution comprising:
a) 40 mg/mL anti-antibody, or antigen binding fragment thereof;
b) about 28-29 mg/mL sucrose;
c) about 0.08 mg/mL polysorbate 80; and
d) about 4 mM histidine buffer at pH 5.0-6.0.

13. The formulation of any one of the above claims, wherein the antibody, or antigen binding fragment thereof, comprises:
(a) a light chain comprising the amino acid sequence of SEQ ID NO:1 and a heavy chain comprising the amino acid sequence of SEQ ID NO:2; or
(b) a light chain variable region comprising the CDR sequences of SEQ ID NOs: 3, 4 and 5 and a heavy chain variable region comprising the CDR sequences of SEQ ID NOs: 6, 7 and 8.

14. A method of treating allergic disease in a mammalian subject in need thereof comprising: administering an effective amount of the formulation of any one of claims 1-12.

15. The formulation of any one of the above claims, wherein the formulation is stable at ° 5 C. for at least 12 months, 18 months, 24 months or 36 months.

16. The formulation of any one of the above claims, wherein the anti-TSLP antibody comprises SEQ ID NO:3, SEQ ID NO:4, SEQ ID NO:5, SEQ ID NO:6, SEQ ID NO:7 and SEQ ID NO:8.

17. The formulation of any one of the above claims, wherein the anti-TSLP antibody comprises SEQ ID NO:1 and SEQ ID NO:2.

18. The formulation of any one of the above claims, wherein said formulation is stable at a temperature of about ° 5 C.-25° C. for at least 36 months.

19. The formulation of any one of the above claims, wherein said formulation comprises ≧98.5% monomer of anti-TSLP when stored for at least 12 months at a temperature of about ° 5 C.-25° C.

20. The formulation of claim 19, wherein said formulation comprises ≧98.5% monomer of anti-TSLP when stored for at least 12 months at a temperature of about 25° C.

21. The formulation of any one of the above claims, wherein said formulation comprises ≧95.0% monomer of anti-TSLP when stored for at least 36 months at a temperature of about ° 5 C.-25° C.

22. The formulation of claim 21, wherein said formulation comprises ≧95.0% monomer of anti-TSLP when stored for at least 36 months at a temperature of about 25° C.

23. The formulation of any of the above claims, wherein the formulation has a viscosity of less than 4 cP at 20° C. when the antibody is present at a concentration of 100 mg/mL.


SEQUENCE LISTING (8 sequences)

SEQ ID NO: 1 - protein, 214 aa, Artificial Sequence
  antiTSLP light chain
EIVLTQSPGTLSLSPGERATLSCRASQPISISVHWYQQKPGQAPRLLIYFASQSISGIPDRFSGSGSGTDFTLTISRLEPEDFAVYYCQQTFSLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 450 aa, Artificial Sequence
  antiTSLP Heavy Chain
QVQLVQSGAEVKKPGASVKVSCKASGYIFTDYAMHWVRQAPGQGLEWMGTFIPLLDTSDYAQKFQGRVTMTADTSTSTAYMELRSLRSDDTAVYYCARMGVTHSYVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 11 aa, Artificial Sequence
  antiTSLP light chain CDR1
RASQPISISVH

SEQ ID NO: 4 - protein, 7 aa, Artificial Sequence
  antiTSLP light chain CDR2
FASQSIS

SEQ ID NO: 5 - protein, 9 aa, Artificial Sequence
  antiTSLP light chain CDR3
QQTFSLPYT

SEQ ID NO: 6 - protein, 10 aa, Artificial Sequence
  antiTSLP heavy chain CDR1
GYIFTDYAMH

SEQ ID NO: 7 - protein, 17 aa, Artificial Sequence
  antiTSLP heavy chain CDR2
TFIPLLDTSDYAQKFQG

SEQ ID NO: 8 - protein, 11 aa, Artificial Sequence
  antiTSLP heavy chain CDR3
MGVTHSYVMDA